(19) United States
(12) Patent Application Publication
(10) Pub. No.: US 2026/0078430 A1
(43) Pub. Date: Mar. 19, 2026

(54) METHODS FOR DETECTING TARGET NUCLEIC ACIDS USING RNA BLOCKING MOLECULES

(71) Applicant: ADVANCED CELL DIAGNOSTICS, INC., Newark, CA (US)

(72) Inventors: Sonali Anantprakash Deshpande, Newark, CA (US); Han Lu, Newark, CA (US); Aparna Sahajan, Newark, CA (US); Manvir Sambhi, Newark, CA (US); Wei Wei, Newark, CA (US); Xiao-Jun Ma, Newark, CA (US); Bingqing Zhang, Newark, CA (US)

(21) Appl. No.: 19/109,354

(22) PCT Filed: Sep. 7, 2023
(86) PCT No.: PCT/US2023/032191

§ 371 Date: Mar. 6, 2025

Related U.S. Application Data
(60) Provisional application No. 63/374,998, filed on Sep. 8, 2022.

(51) Int. Cl. C12Q 1/682; C12Q 1/6806; C12Q 1/6832; C12Q 1/6841

(52) U.S. Cl. CPC C12Q 1/682; C12Q 1/6806; C12Q 1/6832; C12Q 1/6841

(57) ABSTRACT

Embodiments of the present disclosure include compositions and methods for performing in situ hybridization reactions. In particular, the present disclosure provides RNA blocking molecules that enhance detection of a target RNA molecule (e.g., an mRNA molecule, a microRNA (miRNA) molecule, a small non-coding RNA (sncRNA) molecule, a PIWI-interacting RNA (piRNA) molecule, a small interfering RNA (siRNA) molecule, and/or an anti-sense oligo (ASO) molecule) by reducing binding of a target probe to a non-target RNA molecule in a sample.

### CROSS REFERENCE TO RELATED APPLICATIONS

This application claims priority to and the benefit of U.S. Provisional Patent Application No. 63/374,998 filed Sep. 8, 2022, which is incorporated herein by reference in its entirety and for all purposes.

### FIELD

Embodiments of the present disclosure include compositions and methods for performing in situ hybridization reactions. In particular, the present disclosure provides RNA blocking molecules that enhance detection of a target RNA molecule (e.g., an mRNA molecule, a microRNA (miRNA) molecule, a small non-coding RNA (sncRNA) molecule, a PIWI-interacting RNA (piRNA) molecule, and/or a small interfering RNA (siRNA) molecule) by reducing binding of a target probe to a non-target RNA molecule in a sample.

### BACKGROUND

In situ hybridization (ISH) is a technique that allows for precise detection of a specific segment of a nucleic acid within a histologic section. The underlying basis of ISH is that nucleic acids, if preserved adequately within a histologic specimen, can be detected through the application of a complementary nucleic acid strand to which a reporter molecule is attached. Target nucleic acids can be deoxyribonucleic acid (DNA) or ribonucleic acid (RNA). Different types of RNA exist in a cell, including messenger RNA (mRNA), which encode the amino acid sequence of a polypeptide; transfer RNA (tRNA), which bring amino acids to ribosomes during translation; ribosomal RNA (rRNA), which make up the ribosomes, organelles that translate the mRNA; and sncRNA, which is involved in RNA processing. Specifically, sncRNA, miRNA, siRNA, piRNA, are of significant importance in research, diagnostics, and drug development; however, detection sensitivity for RNAs, especially small RNAs, within a cell or tissue sample is challenging.

For example, miRNAs (single-stranded, non-coding, small RNAs) contain only about 16-22 nucleotides. These RNAs are evolutionarily conserved and regulate gene expression by disrupting post-translational modifications or degradation of mRNA. Erratic expression of a single miRNA can lead to misexpression of multiple genes. Due to their importance in regulation of physiological and pathological process, they are used as a biomarker for various diseases including cancer, autoimmune disorders, and neurodegenerative diseases. RNAscope® is an in situ hybridization (ISH)-based technology that detects small RNAs including the miRNA, siRNA and anti-sense oligonucleotides (ASOs) in cells and tissues. This technology uses ISH probes which bind to miRNAs and reveal cellular and sub-cellular spatial expression patterns of small RNA. Probes for RNAscope® are designed in-silico, and up to 34% are predicted to have off-target binding. Therefore, enhanced detection methods are needed to reduce or eliminate off-target binding in order to provide more accurate diagnostic and therapeutic information.

### SUMMARY

Embodiments of the present disclosure include an RNA blocking molecule that includes at least one RNA blocking domain comprising a non-probe-targeting region and a probe-targeting region. In some embodiments, the non-probe-targeting region and the probe-targeting region are contiguous.

In some embodiments, the non-probe-targeting region of the RNA blocking domain is complementary to a non-probe-targeting region of a target RNA molecule. In some embodiments, the probe-targeting region of the RNA blocking domain is complementary to a portion of a probe-targeting region of a target RNA molecule.

In some embodiments, the target RNA molecule is an mRNA molecule, a microRNA (miRNA) molecule, a small non-coding RNA (sncRNA) molecule, a PIWI-interacting RNA (piRNA) molecule, a small interfering RNA (siRNA) molecule, and/or an anti-sense oligo (ASO).

In some embodiments, the non-probe-targeting region of a target mRNA comprises a non-coding region, and wherein the probe-targeting region of the target mRNA comprises a coding region. In some embodiments, the non-probe-targeting region of a target miRNA comprises a region that is not present in the corresponding mature miRNA molecule, and wherein the probe-targeting region of the target miRNA comprises a region that is present in the corresponding mature miRNA molecule.

In some embodiments, the non-probe-targeting region of a target comprises a vector region as in adeno associated viral (AAV), lentiviral and other viral vectors, and wherein the probe-targeting region of the target comprises the ASO.

In some embodiments, the non-probe-targeting region of the RNA blocking domain is from 2 to 50 nucleotides in length. In some embodiments, the probe-targeting region of the RNA blocking domain is from 2 to 20 nucleotides in length.

In some embodiments, the RNA blocking molecule further comprises a second RNA blocking domain comprising a second non-probe-targeting region and a second probe-targeting region. In some embodiments, the second non-probe-targeting region and the second probe-targeting region are contiguous.

In some embodiments, the first and the second RNA blocking domains comprise the same number of nucleotides. In some embodiments, the non-probe-targeting regions of the first and the second RNA blocking domains comprise the same number of nucleotides. In some embodiments, the probe-targeting regions of the first and the second RNA blocking domains comprise the same number of nucleotides.

In some embodiments, the first and the second RNA blocking domains are joined by a linker region. In some embodiments, the linker region is from 1 to 10 nucleotides in length. In some embodiments, the linker region is from 2 to 8 nucleotides in length. In some embodiments, the linker region is from 2 to 5 nucleotides in length. In some embodiments, the linker region comprises at least two types of nucleotides. In some embodiments, the linker region comprises one type of nucleotide. In some embodiments, the linker region consists of thymine nucleotides. In some embodiments, the linker region consists of 1 to 10 thymine nucleotide(s).

In some embodiments, the linker region comprises at least one nucleotide that is non-complementary to a portion of a probe-targeting region of a target RNA molecule. In some embodiments, the linker region comprises from 1 to 10 nucleotides that are non-complementary to a portion of a probe-targeting region of a target RNA molecule. In some embodiments, from about 20% to about 80% of the nucleotides comprising the linker region are non-complementary with a portion of a probe-targeting region of a target RNA molecule.

In some embodiments, the total length of the RNA blocking molecule is from 15 to 100 nucleotides.

Embodiments of the present disclosure also include a kit comprising any of the RNA blocking molecules described herein.

In some embodiments, the kit further comprises at least one target probe that specifically hybridizes to a probe-targeting region of a target RNA molecule. In some embodiments, kit further comprises one or more target probe sets, and each target probe set comprises a pair of target probes that specifically hybridize to a probe-targeting region of a target RNA molecule.

In some embodiments, the kit comprises a reagent for permeabilizing cells, a cross-linking reagent, and/or a protease.

In some embodiments, the kit comprises one or more components of a signal generating complex. In some embodiments, the components of a signal generating complex include: (i) at least one label and at least one label probe, wherein the at least one label probe is capable of hybridizing to the at least one target probe; or (ii) at least one label, at least one label probe, and at least one amplifier hybridized to the at least one label probe and capable of hybridizing to the at least one target probe; or (iii) at least one label, at least one label probe, at least one amplifier hybridized to the at least one label probe, and at least one preamplifier hybridized to the at least one amplifier and capable of hybridizing to the target probe.

In some embodiments, the kit comprises instructions for performing an in situ hybridization reaction.

Embodiments of the present disclosure also include a composition comprising any of the RNA blocking molecules described herein.

In some embodiments, the composition comprises a hybridization buffer. In some embodiments, the composition comprises a reagent for permeabilizing cells, a cross-linking reagent, and/or a protease.

In some embodiments, the composition comprises a biological sample. In some embodiments, the biological sample is a tissue specimen or is derived from a tissue specimen. In some embodiments, the biological sample is a blood sample or is derived from a blood sample. In some embodiments, the biological sample is a cytological sample or is derived from a cytological sample. In some embodiments, the biological sample is cultured cells or a sample containing exosomes.

In some embodiments, the composition comprises at least one target probe that specifically hybridizes to a probe-targeting region of a target RNA molecule in the biological sample.

In some embodiments, the composition comprises one or more components of a signal generating complex. In some embodiments, the components of a signal generating complex include: (i) at least one label and at least one label probe, wherein the at least one label probe is capable of hybridizing to the at least one target probe; or (ii) at least one label, at least one label probe, and at least one amplifier hybridized to the at least one label probe and capable of hybridizing to the at least one target probe; or (iii) at least one label, at least one label probe, at least one amplifier hybridized to the at least one label probe, and at least one preamplifier hybridized to the at least one amplifier and capable of hybridizing to the target probe.

Embodiments of the present disclosure also include a method for performing an in situ hybridization reaction using any of the RNA blocking molecules described herein, any of the kits described herein, and/or any of the compositions described herein.

Embodiments of the present disclosure also include a method of enhancing signal efficiency in an in situ hybridization reaction. In accordance with these embodiments, the method includes contacting a biological sample comprising a target RNA molecule with any of the RNA blocking molecules described herein; contacting the biological sample with at least one target probe that specifically hybridizes to a probe-targeting region of the target RNA molecule; and contacting the biological sample with a signal generating complex to detect the target RNA molecule. In some embodiments of the method, signal efficiency for the target RNA molecule is enhanced as compared to an in situ hybridization reaction that does not comprise contacting the biological sample with the RNA blocking molecules. In some embodiments of the method, enhancing signal efficiency comprises reducing binding of the target probe to non-target RNA molecules in the sample.

In some embodiments of the method, the target RNA molecule is an mRNA molecule, a microRNA (miRNA) molecule, a small non-coding RNA (sncRNA) molecule, a PIWI-interacting RNA (piRNA) molecule, and/or a small interfering RNA (siRNA) molecule.

In some embodiments of the method, the biological sample is a tissue specimen or is derived from a tissue specimen, a blood sample or is derived from a blood sample, or a cytological sample or is derived from a cytological sample.

In some embodiments of the method, the signal generating complex includes: (i) at least one label and at least one label probe, wherein the at least one label probe is capable of hybridizing to the at least one target probe; or (ii) at least one label, at least one label probe, and at least one amplifier hybridized to the at least one label probe and capable of hybridizing to the at least one target probe; or (iii) at least one label, at least one label probe, at least one amplifier hybridized to the at least one label probe, and at least one preamplifier hybridized to the at least one amplifier and capable of hybridizing to the target probe.

In some embodiments, the method comprises treating the biological sample with a reagent for permeabilizing cells, a cross-linking reagent, and/or a protease.

### BRIEF DESCRIPTION OF THE FIGURES

FIGS. 1A-1C: Representative RNA detection blocking strategies tested in accordance with the various embodiments of the present disclosure (FIG. 1A). To test the efficiency of the RNA blocking molecules of the present disclosure for reducing off-target binding of target probes, oligos were designed against a low expressing gene, PPIB as proxy off-target molecule. Four different blocking strategies/designs were tested to determine reduction in signal from PPIB (FIG. 1A). Oligos with complementary sequences to the two sides of the probe design region connected with a T-linker (“Double Competitive Blocking 2” strategy) were most efficient in blocking signal from PPIB (FIG. 1B). Similar oligos were designed and tested against a high-expressing gene, UBC. RNA blocking molecules designed using the same strategy (“Double Competitive Blocking 2” strategy) efficiently reduced the signal from UBC probe (FIG. 1C).

FIGS. 2A-2B: Representative results demonstrating that RNA blocking molecules of the present disclosure directed against pri-miR-21 and pre-miR-205 do not affect detection of miRNA-21 (FIG. 2A) and miRNA-205 (FIG. 2B), respectively.

FIG. 3: Representative results demonstrating that RNA blocking molecules of the present disclosure effectively reduced signal from pre-and pri-miRNA21 (FIG. 3).

FIGS. 4A-4C: Representative results demonstrating that the efficiency of the RNA blockers of the present disclosure varies with varying lengths of overlapping regions (probe-targeting regions), with longer overlap lengths being somewhat more efficient, for both probes targeting UBC (FIG. 4A), and pri-miR-21 (FIGS. 4B and 4C).

FIGS. 5A-5F: Representative results demonstrating that the efficiency of the RNA blockers of the present disclosure varies with varying linker lengths (linkers couple two RNA blocking molecules). Shorter linker lengths (e.g., T linker lengths) are somewhat more efficient (FIG. 5A), with linkers of about 1-3 nucleotides in length being most efficient (FIGS. 5B and 5C). Results also demonstrate that T linkers are somewhat more efficient than linkers composed of other nucleotides (FIG. 5D), and that overall RNA blocker concentration is most efficient from about 20 nM to about 30 nM (FIG. 5E). Taken together, one representative optimized RNA blocker design is provided (FIG. 5F).

### DETAILED DESCRIPTION

Embodiments of the present disclosure include compositions and methods for performing in situ hybridization (ISH) reactions. In particular, the present disclosure provides RNA blocking molecules that enhance detection of a target RNA molecule (e.g., an mRNA molecule, a microRNA (miRNA) molecule, a small non-coding RNA (sncRNA) molecule, a PIWI-interacting RNA (piRNA) molecule, and/or a small interfering RNA (siRNA) molecule) by reducing binding of a target probe to a non-target RNA molecule in a sample. In accordance with these embodiments, the present disclosure provides methods for enhancing signal efficiency for ISH reactions using the RNA blocking molecules described further herein.

### Definitions

As used herein, the term “fixation” or “fixing” when made in reference to fixing a biological sample in the in situ hybridization process refers to a procedure to preserve a biological sample from decay due to, e.g., autolysis or putrefaction. It terminates any ongoing biochemical reactions and may also increase the treated tissues'mechanical strength or stability.

As used herein, the term “one or more” refers to, for example, 1 or more, 2 or more, 3 or more, 4 or more, 5 or more, 6 or more, 7 or more, 8 or more, 9 or more, 10 or more, 20 or more, 30 or more, 40 or more, 50 or more, or a greater number, if desired for a particular use.

The terms “detecting” as used herein generally refer to any form of measurement, and include determining whether an element is present or not. This term includes quantitative and/or qualitative determinations.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids (e.g., can participate in Watson-Crick base pairing interactions). As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

As used herein, the terms “non-coding,” “non-coding region,” or “non-coding sequence” with respect to an RNA molecule generally refer to an RNA sequence/region that is not translated into an amino acid sequence and does not encode a protein, which is in contract to a “coding” sequence/region, which is translated into an amino acid sequence. Generally, non-coding regions of an mRNA molecule include introns, promoters, enhancers, 5′UTRs, 3′UTRs, and the like. Additionally, other species of RNA molecules comprise (or are comprised of) non-coding sequences/regions, including but not limited to, microRNA (miRNA), small non-coding RNA (sncRNA), PIWI-interacting RNA (piRNA), and small interfering RNA (siRNA).

As used herein, the terms “small non-coding RNA” or “sncRNA” generally refer to a large family of RNA molecules (e.g., 18 to 200 nt long) that regulate cell function. Small ncRNAs are involved in nearly all developmental and pathological processes in mammals. While the exact function of many ncRNAs remain unknown, numerous studies have revealed the direct involvement of various small ncRNAs in regulation of gene expression at the levels of posttranscriptional mRNA processing and ribosome biogenesis. Mammalian cells express several classes of small ncRNA, including microRNA (miRNA), small interfering RNAs (siRNA), small nucleolar RNAs (snoRNA), small nuclear RNA (snRNA), PIWI-interacting RNA (piRNA), and tRNA-derived small RNAs (tRFs).

As used herein, the terms “small interfering RNA” or “siRNA” generally refer to small inhibitory RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27 mer) instead of a product (21 mer) and that this improves the rate or efficiency of entry of the siRNA duplex into RISC.

As used herein, the terms “microRNA,” “miRNA,” and “miR” generally refer to a collection of non-coding single-stranded RNA molecules of about 19-28 nucleotides in length, which regulate gene expression. miRNAs are found in a wide range of organisms, including viruses, and have been shown to play a role in development, homeostasis, and disease etiology. Initially the pre-miRNA is present as a long non-perfect double-stranded stem loop RNA that is further processed by Dicer into a siRNA-like duplex, comprising the mature guide strand (miRNA) and a similar-sized fragment known as the passenger strand (miRNA). The miRNA and miRNA may be derived from opposing arms of the pri-miRNA and pre-miRNA. miRNA sequences may be found in libraries of cloned miRNAs but typically at lower frequency than the miRNAs. Although initially present as a double-stranded species with miRNA, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA duplexes, binding site of the target gene, activity of miRNA (repress or activate), and which strand of the miRNA/miRNA duplex is loaded into the RISC. When the miRNA strand of the miRNA:miRNA duplex is loaded into the RISC, the miRNA is removed and degraded. The strand of the miRNA:miRNA duplex that is loaded into the RISC is the strand whose 5′ end is less tightly paired. In cases where both ends of the miRNA:miRNA have roughly equivalent 5′ pairing, both miRNA and miRNA may have gene silencing activity. The RISC identifies target nucleic acids based on high levels of complementarity between the miRNA and the mRNA, especially by nucleotides 2-8 of the miRNA (referred to as “seed sequence”).

As used herein, the terms “piwi-interacting RNA” or “piRNA” generally refer to a class of small noncoding RNAs whose primary function in mammals is silencing germline-resident retrotransposon. PIWI-interacting RNAs (piRNAs) are single-stranded ncRNAs of 26-31 nucleotides that interact with P-element-induced wimpy testis (PIWI) proteins, a germ line-specific Argonaute family. piRNAs display a very diverse set of nucleotide sequences when compared with any other known cellular RNA family, comprising also the largest known class of ncRNAs. piRNAs have been shown to be implicated in the silencing of retrotransposons, both at the post-transcriptional and epigenetic levels, as well as of other genetic elements in germ lines, particularly those during spermatogenesis. They are 5′ monophosphated and 2′-O-methyl modified in the 3′ terminal, characteristics which have been proposed to increase piRNA stability.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

The term “sample” as used herein relates to a material or mixture of materials containing one or more components of interest. The term “sample” includes “biological sample” which refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, cells, and exosomes isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include, but are not limited to, whole blood, partially purified blood, PBMC, tissue biopsies, and the like.

The term “probe” as used herein refers to a capture agent that is directed to a specific target mRNA sequence. Accordingly, each probe of a probe set has a respective target mRNA sequence. In some embodiments, the probe provided herein is a “nucleic acid probe” or “oligonucleotide probe” which refers to a nucleic acid capable of binding to a target nucleic acid of complementary sequence, such as the mRNA biomarkers provided herein, usually through complementary base pairing by forming hydrogen bond. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. The probes can be directly or indirectly labeled with tags, for example, chromophores, lumiphores, or chromogens. By assaying for the presence or absence of the probe, one can detect the presence or absence of a target mRNA biomarker of interest.

As used herein, the term “endogenous” refers to the substances originating from within an organism. As used herein, the term “exogenous” refers to the substances originating from outside an organism.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### RNA Blocking Molecules

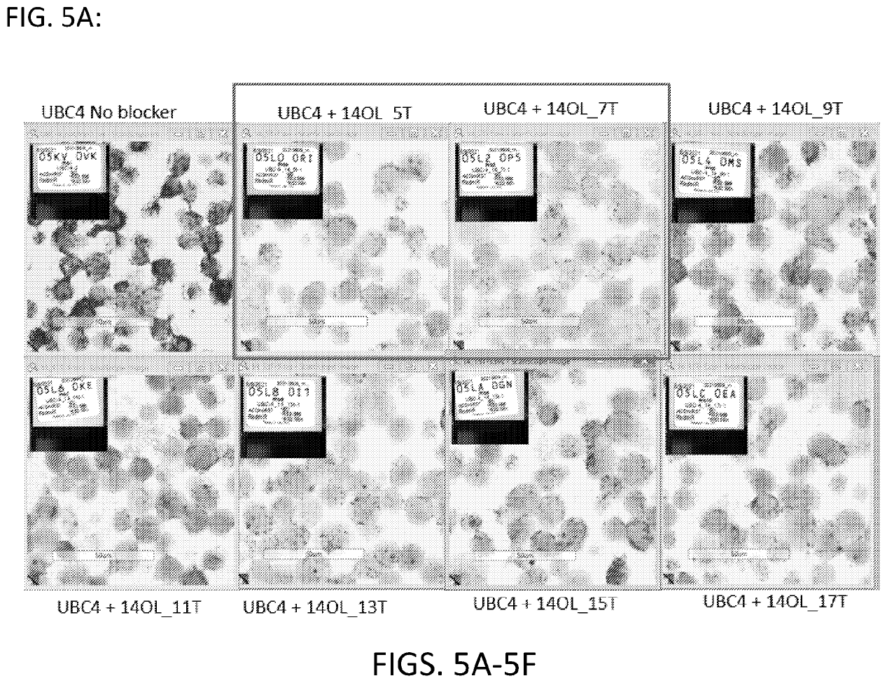

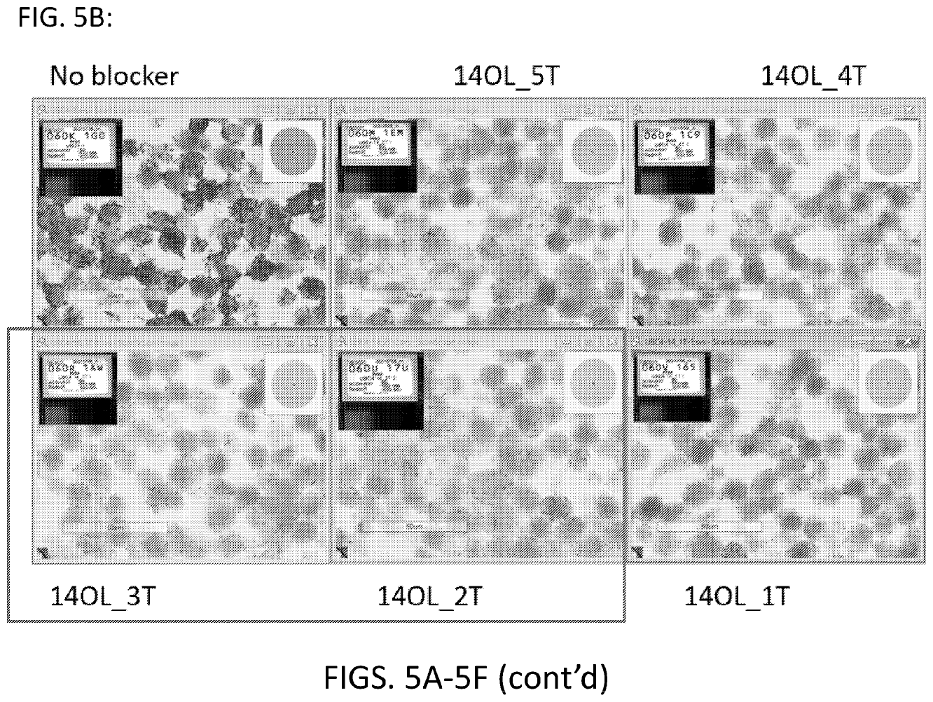

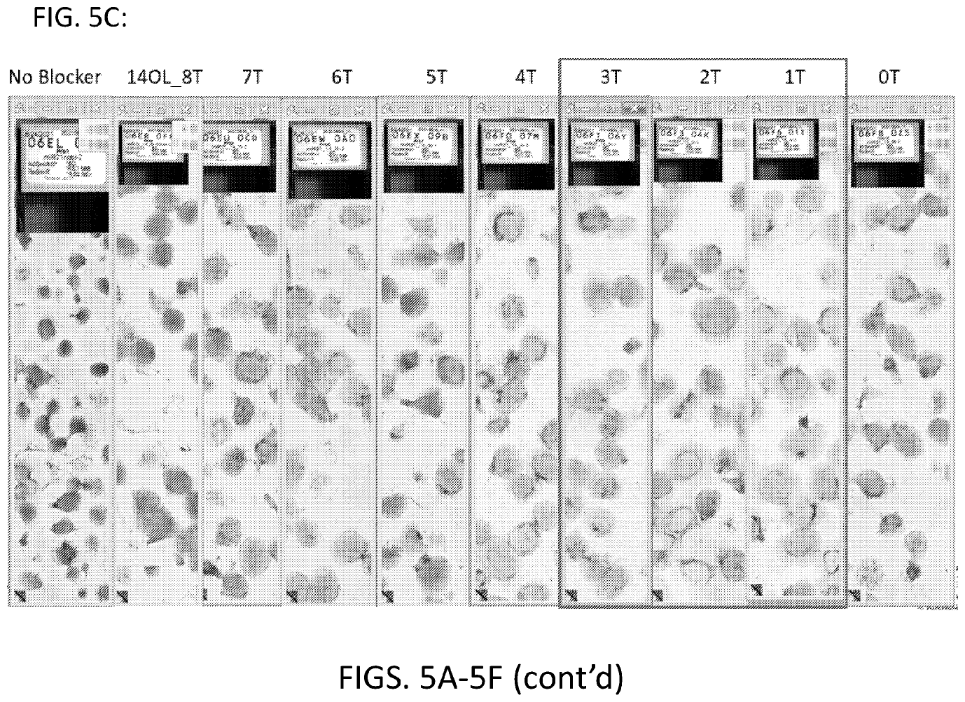

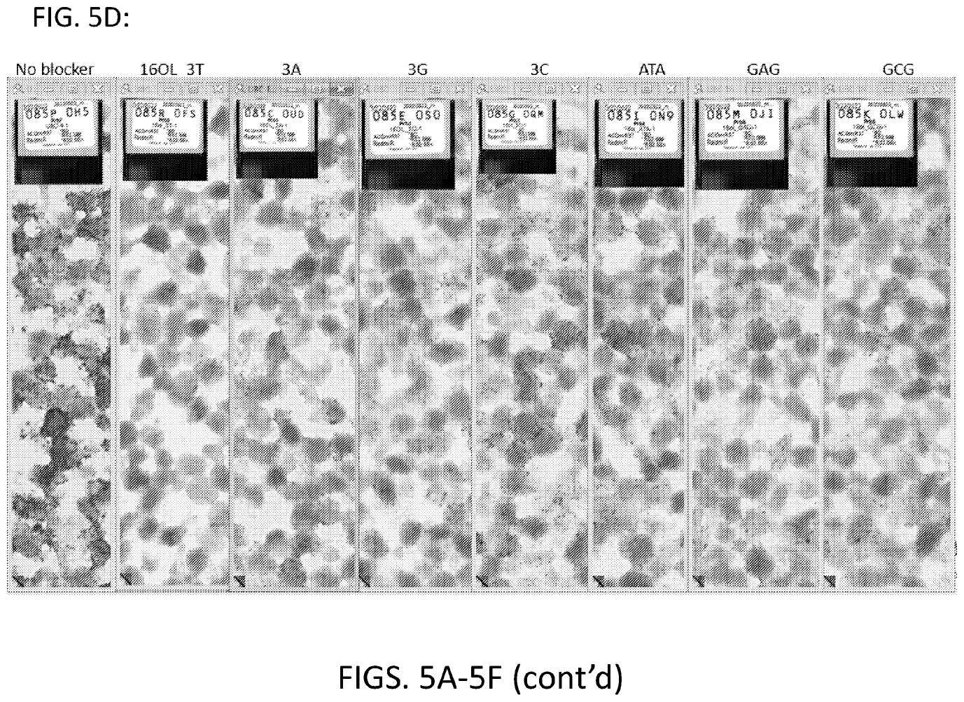

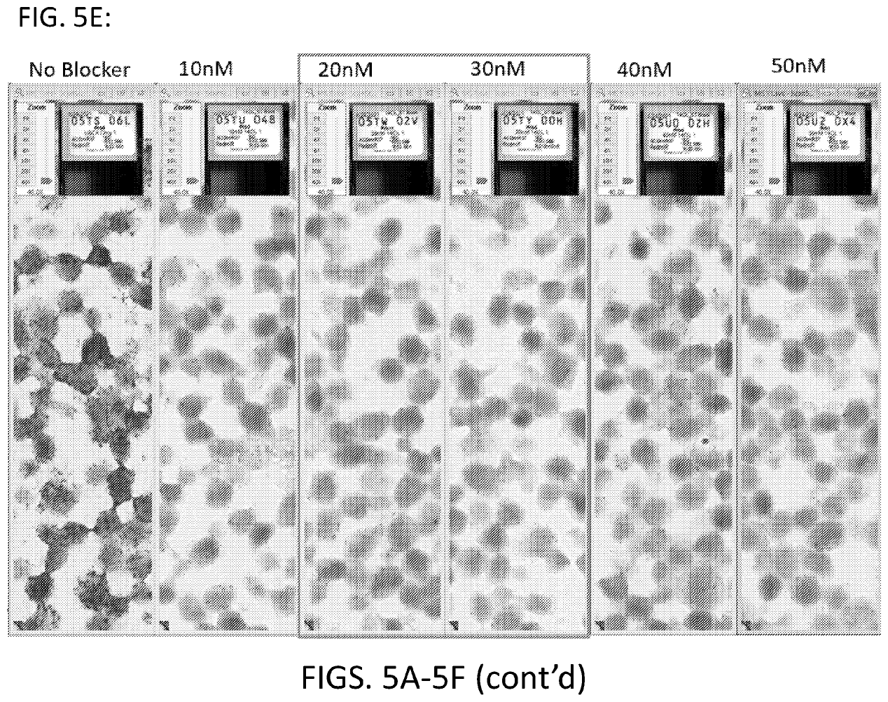

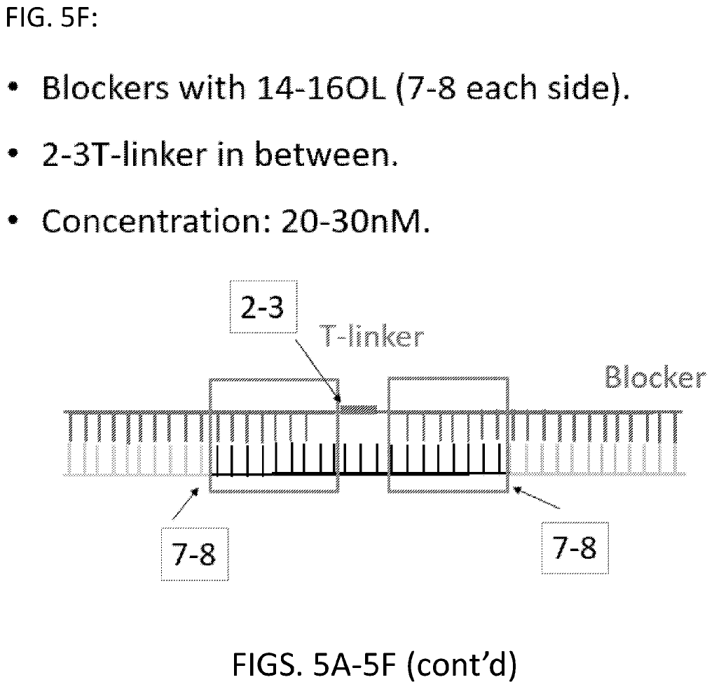

Embodiments of the present disclosure include an RNA blocking molecule, or a plurality of RNA blocking molecules, which includes at least one RNA blocking domain comprising a non-probe-targeting region and a probe-targeting region. The non-probe-targeting region of the RNA blocking domain is generally the region that does not overlap with the region of the target that is designed to be bound by a target probe (see, e.g., FIG. 5F). Conversely, the probe-targeting region of the RNA blocking domain is generally the region that does overlap with the region of the target that is designed to be bound by a target probe (see, e.g., FIG. 5F). In some embodiments, the non-probe-targeting region and the probe-targeting region are contiguous. In accordance with these embodiments, the RNA blocking molecules of the present disclosure can bind to non-target molecules and reduce non-specific binding of the target probe, thereby enhancing signal efficiency.

In some embodiments, the non-probe-targeting region of the RNA blocking domain is complementary to a non-probe-targeting region of a target RNA molecule. In some embodiments, the non-probe-targeting region of the RNA blocking domain is at least 95% complementary to a non-probe-targeting region of a target RNA molecule. In some embodiments, the non-probe-targeting region of the RNA blocking domain is at least 90% complementary to a non-probe-targeting region of a target RNA molecule. In some embodiments, the non-probe-targeting region of the RNA blocking domain is at least 85% complementary to a non-probe-targeting region of a target RNA molecule. In some embodiments, the non-probe-targeting region of the RNA blocking domain is at least 80% complementary to a non-probe-targeting region of a target RNA molecule.

In some embodiments, the probe-targeting region of the RNA blocking domain is complementary to a portion of a probe-targeting region of a target RNA molecule. In some embodiments, the probe-targeting region of the RNA blocking domain is at least 95% complementary to a portion of a probe-targeting region of a target RNA molecule. In some embodiments, the probe-targeting region of the RNA blocking domain is at least 90% complementary to a portion of a probe-targeting region of a target RNA molecule. In some embodiments, the probe-targeting region of the RNA blocking domain is at least 85% complementary to a portion of a probe-targeting region of a target RNA molecule. In some embodiments, the probe-targeting region of the RNA blocking domain is at least 80% complementary to a portion of a probe-targeting region of a target RNA molecule.

In some embodiments, the RNA blocking molecules of the present disclosure are particularly useful for enhancing the detection of target RNA molecules, including target RNA molecules that are smaller in size. In some embodiments, the target RNA molecule includes, but is not limited to, an mRNA molecule, a microRNA (miRNA) molecule, a small non-coding RNA (sncRNA) molecule, a PIWI-interacting RNA (piRNA) molecule, and/or a small interfering RNA (siRNA) molecule. In some embodiments, the RNA blocking molecules of the present disclosure can also be used to enhance detection of DNA molecules, such as anti-sense oligonucleotides (ASOs).

In some embodiments, the RNA blocking molecules of the present disclosure enhance the detection of one or more target messenger RNA (mRNA) molecules in a sample (e.g., cell or tissue sample), such as the detection of target messenger mRNA using in situ hybridization (ISH). In accordance with these embodiments, the non-probe-targeting region of a target mRNA comprises a non-coding region, and the probe-targeting region of the target mRNA comprises a coding region. In other embodiments, the RNA blocking molecules of the present disclosure enhance the detection of one or more target microRNA (miRNA or miR) molecules in a sample (e.g., cell or tissue sample), such as the detection of target miRNA using in situ hybridization (ISH). In accordance with these embodiments, the non-probe-targeting region of a target miRNA comprises a region that is not present in the corresponding mature miRNA molecule (i.e., pr-microRNA or pre-microRNA), and the probe-targeting region of the target miRNA comprises a region that is present in the corresponding mature miRNA molecule.

In some embodiments, the non-probe-targeting region of the RNA blocking domain is from 2 to 50 nucleotides in length. In some embodiments, the non-probe-targeting region of the RNA blocking domain is from 2 to 45 nucleotides in length. In some embodiments, the non-probe-targeting region of the RNA blocking domain is from 2 to 40 nucleotides in length. In some embodiments, the non-probe-targeting region of the RNA blocking domain is from 2 to 35 nucleotides in length. In some embodiments, the non-probe-targeting region of the RNA blocking domain is from 2 to 30 nucleotides in length. In some embodiments, the non-probe-targeting region of the RNA blocking domain is from 2 to 25 nucleotides in length. In some embodiments, the non-probe-targeting region of the RNA blocking domain is from 2 to 20 nucleotides in length. In some embodiments, the non-probe-targeting region of the RNA blocking domain is from 2 to 15 nucleotides in length. In some embodiments, the non-probe-targeting region of the RNA blocking domain is from 2 to 10 nucleotides in length. In some embodiments, the non-probe-targeting region of the RNA blocking domain is from 5 to 50 nucleotides in length. In some embodiments, the non-probe-targeting region of the RNA blocking domain is from 10 to 50 nucleotides in length. In some embodiments, the non-probe-targeting region of the RNA blocking domain is from 15 to 50 nucleotides in length. In some embodiments, the non-probe-targeting region of the RNA blocking domain is from 20 to 50 nucleotides in length. In some embodiments, the non-probe-targeting region of the RNA blocking domain is from 25 to 50 nucleotides in length. In some embodiments, the non-probe-targeting region of the RNA blocking domain is from 30 to 50 nucleotides in length. In some embodiments, the non-probe-targeting region of the RNA blocking domain is from 35 to 50 nucleotides in length. In some embodiments, the non-probe-targeting region of the RNA blocking domain is from 40 to 50 nucleotides in length. In some embodiments, the non-probe-targeting region of the RNA blocking domain is from 45 to 50 nucleotides in length. In some embodiments, the non-probe-targeting region of the RNA blocking domain is from 10 to 40 nucleotides in length. In some embodiments, the non-probe-targeting region of the RNA blocking domain is from 10 to 30 nucleotides in length. In some embodiments, the non-probe-targeting region of the RNA blocking domain is from 15 to 35 nucleotides in length. In some embodiments, the non-probe-targeting region of the RNA blocking domain is from 20 to 40 nucleotides in length.

In some embodiments, the probe-targeting region of the RNA blocking domain is from 2 to 20 nucleotides in length. In some embodiments, the probe-targeting region of the RNA blocking domain is from 5 to 20 nucleotides in length. In some embodiments, the probe-targeting region of the RNA blocking domain is from 10 to 20 nucleotides in length. In some embodiments, the probe-targeting region of the RNA blocking domain is from 15 to 20 nucleotides in length. In some embodiments, the probe-targeting region of the RNA blocking domain is from 2 to 15 nucleotides in length. In some embodiments, the probe-targeting region of the RNA blocking domain is from 2 to 10 nucleotides in length. In some embodiments, the probe-targeting region of the RNA blocking domain is from 2 to 5 nucleotides in length. In some embodiments, the probe-targeting region of the RNA blocking domain is from 10 to 15 nucleotides in length.

In some embodiments, the RNA blocking molecules of the present disclosure further comprise a second RNA blocking domain. In accordance with these embodiments, the second RNA blocking domain of the RNA blocking molecule includes a second non-probe-targeting region and a second probe-targeting region, as described further herein. In some embodiments, the second non-probe-targeting region and the second probe-targeting region are contiguous. In some embodiments, the first and the second RNA blocking domains comprise the same number of nucleotides. In some embodiments, the first and the second RNA blocking domains comprise a different number of nucleotides. In some embodiments, the non-probe-targeting regions of the first and the second RNA blocking domains comprise the same number of nucleotides. In some embodiments, the non-probe-targeting regions of the first and the second RNA blocking domains comprise a different number of nucleotides. In some embodiments, the probe-targeting regions of the first and the second RNA blocking domains comprise the same number of nucleotides. In some embodiments, the probe-targeting regions of the first and the second RNA blocking domains comprise a different number of nucleotides.

In some embodiments, the first and the second RNA blocking domains are joined by a linker region. In some embodiments, the linker region is from 1 to 10 nucleotides in length. In some embodiments, the linker region is from 2 to 8 nucleotides in length. In some embodiments, the linker region is from 2 to 5 nucleotides in length. In some embodiments, the linker region comprises at least two types of nucleotides. In some embodiments, the linker region comprises one type of nucleotide. In some embodiments, the linker region consists of thymine nucleotides. In some embodiments, the linker region consists of 1 to 10 thymine nucleotide(s).

In some embodiments, the linker region comprises at least one nucleotide that is non-complementary to a portion of a probe-targeting region of a target RNA molecule. For example, if the linker region comprises thymine nucleotides, then at least one nucleotide in the probe-targeting region of a target RNA molecule comprises a cytosine or guanine. In some embodiments, the linker region comprises from 1 to 10 nucleotides that are non-complementary to a portion of a probe-targeting region of a target RNA molecule. In some embodiments, the linker region comprises from 2 to 10 nucleotides that are non-complementary to a portion of a probe-targeting region of a target RNA molecule. In some embodiments, the linker region comprises from 3 to 10 nucleotides that are non-complementary to a portion of a probe-targeting region of a target RNA molecule. In some embodiments, the linker region comprises from 4 to 10 nucleotides that are non-complementary to a portion of a probe-targeting region of a target RNA molecule. In some embodiments, the linker region comprises from 5 to 10 nucleotides that are non-complementary to a portion of a probe-targeting region of a target RNA molecule. In some embodiments, the linker region comprises from 6 to 10 nucleotides that are non-complementary to a portion of a probe-targeting region of a target RNA molecule. In some embodiments, the linker region comprises from 7 to 10 nucleotides that are non-complementary to a portion of a probe-targeting region of a target RNA molecule. In some embodiments, the linker region comprises from 8 to 10 nucleotides that are non-complementary to a portion of a probe-targeting region of a target RNA molecule. In some embodiments, the linker region comprises from 9 to 10 nucleotides that are non-complementary to a portion of a probe-targeting region of a target RNA molecule. In some embodiments, the linker region comprises from 1 to 9 nucleotides that are non-complementary to a portion of a probe-targeting region of a target RNA molecule. In some embodiments, the linker region comprises from 1 to 8 nucleotides that are non-complementary to a portion of a probe-targeting region of a target RNA molecule. In some embodiments, the linker region comprises from 1 to 7 nucleotides that are non-complementary to a portion of a probe-targeting region of a target RNA molecule. In some embodiments, the linker region comprises from 1 to 6 nucleotides that are non-complementary to a portion of a probe-targeting region of a target RNA molecule. In some embodiments, the linker region comprises from 1 to 5 nucleotides that are non-complementary to a portion of a probe-targeting region of a target RNA molecule. In some embodiments, the linker region comprises from 1 to 4 nucleotides that are non-complementary to a portion of a probe-targeting region of a target RNA molecule. In some embodiments, the linker region comprises from 1 to 3 nucleotides that are non-complementary to a portion of a probe-targeting region of a target RNA molecule. In some embodiments, the linker region comprises from 1 to 2 nucleotides that are non-complementary to a portion of a probe-targeting region of a target RNA molecule. In some embodiments, the linker region comprises from 2 to 8 nucleotides that are non-complementary to a portion of a probe-targeting region of a target RNA molecule. In some embodiments, the linker region comprises from 4 to 6 nucleotides that are non-complementary to a portion of a probe-targeting region of a target RNA molecule. In some embodiments, the linker region comprises from 2 to 3 nucleotides that are non-complementary to a portion of a probe-targeting region of a target RNA molecule. In some embodiments, the linker region comprises from 5 to 7 nucleotides that are non-complementary to a portion of a probe-targeting region of a target RNA molecule.

In some embodiments, from about 20% to about 80% of the nucleotides comprising the linker region are non-complementary with a portion of a probe-targeting region of a target RNA molecule. In some embodiments, from about 25% to about 80% of the nucleotides comprising the linker region are non-complementary with a portion of a probe-targeting region of a target RNA molecule. In some embodiments, from about 30% to about 80% of the nucleotides comprising the linker region are non-complementary with a portion of a probe-targeting region of a target RNA molecule. In some embodiments, from about 35% to about 80% of the nucleotides comprising the linker region are non-complementary with a portion of a probe-targeting region of a target RNA molecule. In some embodiments, from about 40% to about 80% of the nucleotides comprising the linker region are non-complementary with a portion of a probe-targeting region of a target RNA molecule. In some embodiments, from about 45% to about 80% of the nucleotides comprising the linker region are non-complementary with a portion of a probe-targeting region of a target RNA molecule. In some embodiments, from about 50% to about 80% of the nucleotides comprising the linker region are non-complementary with a portion of a probe-targeting region of a target RNA molecule. In some embodiments, from about 55% to about 80% of the nucleotides comprising the linker region are non-complementary with a portion of a probe-targeting region of a target RNA molecule. In some embodiments, from about 60% to about 80% of the nucleotides comprising the linker region are non-complementary with a portion of a probe-targeting region of a target RNA molecule. In some embodiments, from about 65% to about 80% of the nucleotides comprising the linker region are non-complementary with a portion of a probe-targeting region of a target RNA molecule. In some embodiments, from about 70% to about 80% of the nucleotides comprising the linker region are non-complementary with a portion of a probe-targeting region of a target RNA molecule. In some embodiments, from about 75% to about 80% of the nucleotides comprising the linker region are non-complementary with a portion of a probe-targeting region of a target RNA molecule. In some embodiments, from about 20% to about 75% of the nucleotides comprising the linker region are non-complementary with a portion of a probe-targeting region of a target RNA molecule. In some embodiments, from about 20% to about 70% of the nucleotides comprising the linker region are non-complementary with a portion of a probe-targeting region of a target RNA molecule. In some embodiments, from about 20% to about 65% of the nucleotides comprising the linker region are non-complementary with a portion of a probe-targeting region of a target RNA molecule. In some embodiments, from about 20% to about 60% of the nucleotides comprising the linker region are non-complementary with a portion of a probe-targeting region of a target RNA molecule. In some embodiments, from about 20% to about 55% of the nucleotides comprising the linker region are non-complementary with a portion of a probe-targeting region of a target RNA molecule. In some embodiments, from about 20% to about 50% of the nucleotides comprising the linker region are non-complementary with a portion of a probe-targeting region of a target RNA molecule. In some embodiments, from about 20% to about 45% of the nucleotides comprising the linker region are non-complementary with a portion of a probe-targeting region of a target RNA molecule. In some embodiments, from about 20% to about 40% of the nucleotides comprising the linker region are non-complementary with a portion of a probe-targeting region of a target RNA molecule. In some embodiments, from about 20% to about 35% of the nucleotides comprising the linker region are non-complementary with a portion of a probe-targeting region of a target RNA molecule. In some embodiments, from about 20% to about 30% of the nucleotides comprising the linker region are non-complementary with a portion of a probe-targeting region of a target RNA molecule. In some embodiments, from about 20% to about 25% of the nucleotides comprising the linker region are non-complementary with a portion of a probe-targeting region of a target RNA molecule. In some embodiments, from about 30% to about 70% of the nucleotides comprising the linker region are non-complementary with a portion of a probe-targeting region of a target RNA molecule. In some embodiments, from about 40% to about 60% of the nucleotides comprising the linker region are non-complementary with a portion of a probe-targeting region of a target RNA molecule. In some embodiments, from about 50% to about 80% of the nucleotides comprising the linker region are non-complementary with a portion of a probe-targeting region of a target RNA molecule.

In some embodiments, the total length of the RNA blocking molecule is from 15 to 100 nucleotides. In some embodiments, the total length of the RNA blocking molecule is from 20 to 100 nucleotides. In some embodiments, the total length of the RNA blocking molecule is from 25 to 100 nucleotides. In some embodiments, the total length of the RNA blocking molecule is from 30 to 100 nucleotides. In some embodiments, the total length of the RNA blocking molecule is from 35 to 100 nucleotides. In some embodiments, the total length of the RNA blocking molecule is from 40 to 100 nucleotides. In some embodiments, the total length of the RNA blocking molecule is from 45 to 100 nucleotides. In some embodiments, the total length of the RNA blocking molecule is from 50 to 100 nucleotides. In some embodiments, the total length of the RNA blocking molecule is from 55 to 100 nucleotides. In some embodiments, the total length of the RNA blocking molecule is from 60 to 100 nucleotides. In some embodiments, the total length of the RNA blocking molecule is from 65 to 100 nucleotides. In some embodiments, the total length of the RNA blocking molecule is from 70 to 100 nucleotides. In some embodiments, the total length of the RNA blocking molecule is from 75 to 100 nucleotides. In some embodiments, the total length of the RNA blocking molecule is from 80 to 100 nucleotides. In some embodiments, the total length of the RNA blocking molecule is from 85 to 100 nucleotides. In some embodiments, the total length of the RNA blocking molecule is from 90 to 100 nucleotides. In some embodiments, the total length of the RNA blocking molecule is from 95 to 100 nucleotides. In some embodiments, the total length of the RNA blocking molecule is from 15 to 95 nucleotides. In some embodiments, the total length of the RNA blocking molecule is from 15 to 90 nucleotides. In some embodiments, the total length of the RNA blocking molecule is from 15 to 85 nucleotides. In some embodiments, the total length of the RNA blocking molecule is from 15 to 80 nucleotides. In some embodiments, the total length of the RNA blocking molecule is from 15 to 75 nucleotides. In some embodiments, the total length of the RNA blocking molecule is from 15 to 70 nucleotides. In some embodiments, the total length of the RNA blocking molecule is from 15 to 65 nucleotides. In some embodiments, the total length of the RNA blocking molecule is from 15 to 60 nucleotides. In some embodiments, the total length of the RNA blocking molecule is from 15 to 55 nucleotides. In some embodiments, the total length of the RNA blocking molecule is from 15 to 50 nucleotides. In some embodiments, the total length of the RNA blocking molecule is from 15 to 45 nucleotides. In some embodiments, the total length of the RNA blocking molecule is from 15 to 40 nucleotides. In some embodiments, the total length of the RNA blocking molecule is from 15 to 35 nucleotides. In some embodiments, the total length of the RNA blocking molecule is from 15 to 30 nucleotides. In some embodiments, the total length of the RNA blocking molecule is from 15 to 25 nucleotides. In some embodiments, the total length of the RNA blocking molecule is from 15 to 20 nucleotides. In some embodiments, the total length of the RNA blocking molecule is from 20 to 80 nucleotides. In some embodiments, the total length of the RNA blocking molecule is from 30 to 70 nucleotides. In some embodiments, the total length of the RNA blocking molecule is from 40 to 60 nucleotides. In some embodiments, the total length of the RNA blocking molecule is from 50 to 90 nucleotides. In some embodiments, the total length of the RNA blocking molecule is from 30 to 60 nucleotides.

In accordance with these embodiments, the present disclosure provides compositions and methods for performing an in situ hybridization (ISH) reaction to detect a target nucleic acid (e.g., RNA) in a sample using the RNA blocking molecules described herein. In some embodiments, the in situ hybridization detects a target nucleic acid comprising less than 100 nucleotides. In some embodiments, the target nucleic acid comprises 15-100 nucleotides. In some embodiments, the target nucleic acid comprises 15-80 nucleotides. In some embodiments, the target nucleic acid comprises 15-60 nucleotides. In some embodiments, the target nucleic acid comprises 15-50 nucleotides. In some embodiments, the target nucleic acid comprises 15-40 nucleotides. In some embodiments, the target nucleic acid comprises less than 90 nucleotides. In some embodiments, the target nucleic acid comprises less than 80 nucleotides. In some embodiments, the target nucleic acid comprises less than 70 nucleotides. In some embodiments, the target nucleic acid comprises less than 60 nucleotides. In some embodiments, the target nucleic acid comprises less than 50 nucleotides. In some embodiments, the target nucleic acid comprises less than 40 nucleotides. In some embodiments, the target nucleic acid comprises less than 30 nucleotides. In some embodiments, the target nucleic acid comprises less than 20 nucleotides. In some embodiments, the target nucleic acid comprises less than 16 nucleotides. In some embodiments, the in situ hybridization is for detecting DNA. In some embodiments, the in situ hybridization is for detecting RNA. The method provided herein can also be used to detect longer nucleic acid, e.g., comprising more than 100, 200, 300, 500, 1000 or more nucleotides.

In some embodiments, the methods provided herein include detecting small RNA species in a sample. In one embodiment, the RNA detected is less than 100 nucleotides. In one embodiment, the RNA detected is less than 50 nucleotides. In one embodiment, the RNA detected is less than 40 nucleotides. In one embodiment, the RNA detected is between 10 and 40 nucleotides. In one embodiment, the RNA detected is between 15 and 40 nucleotides. In one embodiment, the RNA detected is between 30 and 40 nucleotides. In some embodiments, the methods provided herein detect naturally occurring small nucleic acids. Naturally occurring small nucleic acids play crucial and diverse cellular functions from transcription and RNA processing to translation. A common feature of these RNAs is their size ranging from 15 and 40 nucleotides long. In other embodiments, the methods provided herein detect synthetic nucleic acids.

In one embodiment, the method includes detecting sncRNAs. In one embodiment, the method includes detecting miRNAs. In one embodiment, the method includes detecting siRNAs. In one embodiment, the method includes detecting piRNAs. In one embodiment, the method includes detecting endogenous RNAs. In another embodiment, the method includes detecting exogenous RNAs. sncRNAs have emerged as valuable therapeutics for disease intervention with the ability to efficiently modulate gene expression in clinically relevant model systems (Watts et al., Journal of Pathology, 226(2), 365-379, 2012; Schoch et al., Neuron Review, 94, 1056-1070, 2017). miRNAs are naturally occurring small (˜22 nucleotide) regulatory RNAs present in all multicellular organisms, single-cell alga, and some viruses (Molnar et al., Nature, 447(7148), 2007; Bartel, Cell, 173, 20-51, 2018). To date more than 15,000 miRNAs from animals, plants and viruses have been registered (www.mirbase.org), many expressed in a tissue, cell-type, and cell-state specific manner. Deregulation of miRNA expression may lead to severe conditions, such as neurological disorders, infertility, immune disorders or cancers.

In accordance with these embodiments, the methods and compositions of the present disclosure include performing an in situ hybridization reaction to detect and/or quantify RNA in a biological sample. In one embodiment, the biological sample is a tissue specimen or is derived from a tissue specimen. In one embodiment, the biological sample is a blood sample or is derived from a blood sample. In one embodiment, the biological sample is a cytological sample or is derived from a cytological sample. In one embodiment, the biological sample is cultured cells. In another embodiment, the biological sample is an exosome.

Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the disclosure are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods in the art. The methods of the disclosure can be used in diagnostic applications to identify the presence or absence of pathological cells based on the presence or absence of a nucleic acid target that is a biomarker indicative of a pathology.

It is understood by those skilled in the art based on the present disclosure that any of a number of suitable samples can be used for detecting target nucleic acids using the methods and compositions provided herein. The sample for use in the methods provided herein is generally a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions, cells, and/or exosomes isolated from an organism such as a mammal. Exemplary biological samples include, but are not limited to, a cell culture, including a primary cell culture, a cell line, a tissue, an organ, an organelle, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes.

Collection of cytological samples for analysis by methods provided herein are known in the art (see, for example, Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004).

For example, methods for processing samples for analysis of cervical tissue, including tissue biopsy and cytology samples, are known in the art (see, for example, Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract. Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)).

In some embodiments, the sample is a tissue specimen or is derived from a tissue specimen. In some embodiments, the tissue specimen is formalin-fixed paraffin-embedded (FFPE). In some embodiments, the tissue specimen is fresh frozen. In some embodiments, the tissue specimen is prepared with a fixative other than formalin. In some embodiments, the fixative other than formalin is selected from the group consisting of ethanol, methanol, Bouin's fixative, B5, and I.B.F. In another embodiments, the sample is a blood sample or is derived from a blood sample. In other embodiments, the sample is a cytological sample or is derived from a cytological sample. In some embodiments, the method comprises treating the biological sample with a reagent for permeabilizing cells, a cross-linking reagent, and/or a protease.

### Methods of Detecting a Target Nucleic Acid

Embodiments of the present disclosure also include methods and compositions for performing an in situ hybridization reaction using any of the RNA blocking molecules described herein, any of the kits described herein, and/or any of the compositions described herein. Embodiments of the present disclosure also include a method of enhancing signal efficiency in an in situ hybridization reaction. In accordance with these embodiments, the method includes contacting a biological sample comprising a target RNA molecule with any of the RNA blocking molecules described herein. In some embodiments, the method includes contacting the biological sample with at least one target probe that specifically hybridizes to a probe-targeting region of the target RNA molecule. In some embodiments, the method includes contacting the biological sample with a signal generating complex to detect the target RNA molecule. In some embodiments of the method, signal efficiency for the target RNA molecule is enhanced as compared to an in situ hybridization reaction that does not comprise contacting the biological sample with the RNA blocking molecules. In some embodiments of the method, enhancing signal efficiency comprises reducing binding of the target probe to non-target RNA molecules in the sample.

In some embodiments of the method, the target RNA molecule is an mRNA molecule, a microRNA (miRNA) molecule, a small non-coding RNA (sncRNA) molecule, a PIWI-interacting RNA (piRNA) molecule, and/or a small interfering RNA (siRNA) molecule, as described further herein. In some embodiments of the method, the biological sample is a tissue specimen or is derived from a tissue specimen, a blood sample or is derived from a blood sample, or a cytological sample or is derived from a cytological sample, as described further herein.

In some embodiments of the method, the signal generating complex (SGC) includes at least one label and at least one label probe, wherein the at least one label probe is capable of hybridizing to the at least one target probe. In some embodiments of the method, the SGC includes at least one label, at least one label probe, and at least one amplifier hybridized to the at least one label probe and capable of hybridizing to the at least one target probe. In some embodiments of the method, the SGC includes at least one label, at least one label probe, at least one amplifier hybridized to the at least one label probe, and at least one preamplifier hybridized to the at least one amplifier and capable of hybridizing to the target probe.

In some embodiments, the method provided herein detects relatively short nucleic acid. For example, in some embodiments, the in situ hybridization detects a target nucleic acid comprising less than 100 nucleotides. In some embodiments, the target nucleic acid comprises 15-100 nucleotides. In some embodiments, the target nucleic acid comprises 15-80 nucleotides. In some embodiments, the target nucleic acid comprises 15-60 nucleotides. In some embodiments, the target nucleic acid comprises 15-50 nucleotides. In some embodiments, the target nucleic acid comprises 15-40 nucleotides. In some embodiments, the target nucleic acid comprises less than 90 nucleotides. In some embodiments, the target nucleic acid comprises less than 80 nucleotides. In some embodiments, the target nucleic acid comprises less than 70 nucleotides. In some embodiments, the target nucleic acid comprises less than 60 nucleotides. In some embodiments, the target nucleic acid comprises less than 50 nucleotides. In some embodiments, the target nucleic acid comprises less than 40 nucleotides. In some embodiments, the target nucleic acid comprises less than 30 nucleotides. In some embodiments, the target nucleic acid comprises less than 20 nucleotides. In some embodiments, the target nucleic acid comprises less than 16 nucleotides.

In some embodiments, the in situ hybridization is for detecting small RNA species. In one embodiment, the RNA detected is less than 100 nucleotides. In one embodiment, the RNA detected is less than 50 nucleotides. In one embodiment, the RNA detected is less than 40 nucleotides. In one embodiment, the RNA detected is between 10 and 40 nucleotides. In one embodiment, the RNA detected is between 15 and 40 nucleotides. In one embodiment, the RNA detected is between 30 and 40 nucleotides. In one embodiment, the method is for detecting sncRNAs. In one embodiment, the method is for detecting miRNAs. In one embodiment, the method is for detecting siRNAs. In one embodiment, the method is for detecting piRNAs. In one embodiment, the method is for detecting ASOs. In one embodiment, the method is for detecting endogenous RNAs. In one embodiment, the method is for detecting exogenous RNAs.

In some embodiments, the in situ hybridization provided herein comprises providing at least one set of one or more target probe(s) capable of hybridizing to said target nucleic acid; providing a signal-generating complex capable of hybridizing to said set of one or more target probe(s), said signal-generating complex comprises a nucleic acid component capable of hybridizing to said set of one or more target probe(s) and a label probe; hybridizing said target nucleic acid to said set of one or more target probe(s); and capturing the signal-generating complex to said set of one or more target probe(s) and thereby capturing the signal-generating complex to said target nucleic acid. In some embodiments, each set of one or more target probe(s) comprises a single probe. In other embodiments, each set of one or more target probe(s) comprises two probes. In yet other embodiments, each set of one or more target probe(s) comprises more than two probes. In some embodiments, when each set of target probes comprises a single target probe, a signal-generating complex is formed when the single target probe is bound to the target nucleic acid. In other embodiments, when each set of target probes comprise two target probes, a signal-generating complex is formed when both members of a target probe pair are bound to the target nucleic acid.

In some specific embodiments, the RNA ISH used herein is RNAscope®, which is described in more detail in, e.g., U.S. Pat. Nos. 7,709,198, 8,604,182, and 8,951,726. Specifically, RNAscope® describes using specially designed oligonucleotide probes in combination with a branched-DNA-like signal-generating complex to reliably detect RNA as small as 1 kilobase at single-molecule sensitivity under standard bright-field microscopy (Anderson et al., J. Cell. Biochem. 117(10):2201-2208 (2016); Wang et al., J. Mol. Diagn. 14(1):22-29 (2012)).

In some embodiments, each target probe comprises a target (T) section and a label (L) section, wherein the T section is a nucleic acid sequence complementary to a section on the target nucleic acid and the L section is a nucleic acid sequence complementary to a section on the nucleic acid component of the signal-generating complex, and wherein the T sections of the one or more target probe(s) are complementary to non-overlapping regions of the target nucleic acid, and the L sections of the one or more target probe(s) are complementary to non-overlapping regions of the nucleic acid component of the generating complex.

In some embodiments, one set of one or more target probe(s) is used to detect a target nucleic acid. In other embodiments, two or more sets of one or more target probe(s) are used to detect a target nucleic acid. In some embodiments, two sets of one or more target probe(s) are used to detect a target nucleic acid. In some embodiments, three sets of one or more target probe(s) are used to detect a target nucleic acid. In some embodiments, four sets of one or more target probe(s) are used to detect a target nucleic acid. In some embodiments, five sets of one or more target probe(s) are used to detect a target nucleic acid. In some embodiments, six sets of one or more target probe(s) are used to detect a target nucleic acid. In some embodiments, seven sets of one or more target probe(s) are used to detect a target nucleic acid. In some embodiments, eight sets of one or more target probe(s) are used to detect a target nucleic acid. In some embodiments, nine sets of one or more target probe(s) are used to detect a target nucleic acid. In some embodiments, ten sets of one or more target probe(s) are used to detect a target nucleic acid. In some embodiments, more than 10 sets of one or more target probe(s) are used to detect a target nucleic acid. In some embodiments, more than 15 sets of one or more target probe(s) are used to detect a target nucleic acid. In some embodiments, more than 20 sets of one or more target probe(s) are used to detect a target nucleic acid. In some embodiments, more than 30 sets of one or more target probe(s) are used to detect a target nucleic acid. In some embodiments, the method provided herein is for detecting multiple nucleic acid targets. In some embodiments, the multiple nucleic acid targets all comprise less than 100 nucleotides. In other embodiments, some of the nucleic acid targets comprise less than 100 nucleotides, while other targets comprise more than 100 nucleotides.

As used herein, a “target probe” generally refers to a polynucleotide that is capable of hybridizing to a target nucleic acid and capturing or binding a label probe or signal-generating complex (SGC) component to that target nucleic acid. The target probe can hybridize directly to the label probe, or it can hybridize to one or more nucleic acids that in turn hybridize to the label probe; for example, the target probe can hybridize to an amplifier, a pre-amplifier or a pre-pre-amplifier in an SGC. The target probe thus includes a first polynucleotide sequence that is complementary to a polynucleotide sequence of the target nucleic acid and a second polynucleotide sequence that is complementary to a polynucleotide sequence of the label probe, amplifier, pre-amplifier, pre-pre-amplifier, or the like. The target probe is generally single stranded so that the complementary sequence is available to hybridize with a corresponding target nucleic acid, label probe, amplifier, pre-amplifier or pre-pre-amplifier. In some embodiments, the target probes are provided as a pair.

As used herein, the term “label probe” refers to an entity that binds to a target molecule, directly or indirectly, generally indirectly, and allows the target to be detected. A label probe (or “LP”) contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target nucleic acid. In general, the label probe can hybridize to a nucleic acid that is in turn hybridized to the target nucleic acid or to one or more other nucleic acids that are hybridized to the target nucleic acid. Thus, the label probe can comprise a polynucleotide sequence that is complementary to a polynucleotide sequence, particularly a portion of the target nucleic acid. Alternatively, the label probe can comprise at least one polynucleotide sequence that is complementary to a polynucleotide sequence in an amplifier, pre-amplifier, or pre-pre-amplifier in an SGC.

In some embodiments, the SGC provided herein comprises additional comments such an amplifier, a pre-amplifier, and/or a pre-pre-amplifier. As used herein, an “amplifier” is a molecule, typically a polynucleotide, that is capable of hybridizing to multiple label probes. Typically, the amplifier hybridizes to multiple identical label probes. The amplifier can also hybridize to a target nucleic acid, to at least one target probe of a pair of target probes, to both target probes of a pair of target probes, or to nucleic acid bound to a target probe such as an amplifier, pre-amplifier or pre-pre-amplifier. For example, the amplifier can hybridize to at least one target probe and to a plurality of label probes, or to a pre-amplifier and a plurality of label probes. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. publications 2008/0038725 and 2009/0081688, each of which is incorporated by reference.

As used herein, a “pre-amplifier” is a molecule, typically a polynucleotide, that serves as an intermediate binding component between one or more target probes and one or more amplifiers. Typically, the pre-amplifier hybridizes simultaneously to one or more target probes and to a plurality of amplifiers. Exemplary pre-amplifiers are described, for example, in U.S. Pat. Nos. 5,635,352, 5,681,697 and 7,709,198 and U.S. publications 2008/0038725, 2009/0081688 and 2017/0101672, each of which is incorporated by reference.

As used herein, a “pre-pre-amplifier” is a molecule, typically a polynucleotide, that serves as an intermediate binding component between one or more target probes and one or more pre-amplifiers. Typically, the pre-pre-amplifier hybridizes simultaneously to one or more target probes and to a plurality of pre-amplifiers. Exemplary pre-pre-amplifiers are described, for example, in U.S. Pat. No. 11,078,528, which is incorporated by reference.

A label is typically used in RNA in situ hybridization for detecting target nucleic acid. As used herein, a “label” is a moiety that facilitates detection of a molecule. Common labels include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In a particular embodiment, the label is an enzyme. Exemplary enzyme labels include, but are not limited to horseradish peroxidase (HRP), alkaline phosphatase (AP), β-galactosidase, glucose oxidase, and the like, as well as various proteases. Other labels include, but are not limited to, fluorophores, dinitrophenyl (DNP), and the like. Labels are known to those skilled in the art, as described, for example, in Hermanson, Bioconjugate Techniques, Academic Press, San Diego (1996), and U.S. Pat. Nos. 3,817,837; 3,850,752; 3,939,350; 3,996,345; 4,277,437; 4,275,149; and 4,366,241. Many labels are commercially available and can be used in methods and assays of the disclosure, including detectable enzyme/substrate combinations (Pierce, Rockford IL; Santa Cruz Biotechnology, Dallas TX; Life Technologies, Carlsbad CA). In a particular embodiment of the disclosure, the enzyme can utilize a chromogenic or fluorogenic substrate to produce a detectable signal, as described herein. Exemplary labels are described herein.

Any of a number of enzymes or non-enzyme labels can be utilized so long as the enzymatic activity or non-enzyme label, respectively, can be detected. The enzyme thereby produces a detectable signal, which can be utilized to detect a target nucleic acid. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have known chromogenic or fluorogenic substrates include various peptidases, where chromogenic or fluorogenic peptide substrates can be utilized to detect proteolytic cleavage reactions. The use of chromogenic and fluorogenic substrates is also known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods provided herein.

Various chromogenic or fluorogenic substrates to produce detectable signals are known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-nitrophenyl phosphate (PNPP) for alkaline phosphatase; 1-methyl-3-indolyl-β-D-galactopyranoside and 2-methoxy-4-(2-nitrovinyl)phenyl β-D-galactopyranoside for β-galactosidase; 2-methoxy-4-(2-nitrovinyl)phenyl β-D-glucopyranoside for β-glucosidase; and the like. Exemplary fluorogenic substrates include, but are not limited to, 4-(trifluoromethyl)umbelliferyl phosphate for alkaline phosphatase; 4-methylumbelliferyl phosphate bis (2-amino-2-methyl-1,3-propanediol), 4-methylumbelliferyl phosphate bis (cyclohexylammonium) and 4-methylumbelliferyl phosphate for phosphatases; QuantaBlu™ and Quintolet for horseradish peroxidase; 4-methylumbelliferyl β-D-galactopyranoside, fluorescein di(β-D-galactopyranoside) and naphthofluorescein di-(β-D-galactopyranoside) for β-galactosidase; 3-acetylumbelliferyl β-D-glucopyranoside and 4-methylumbelliferyl-β-D-glucopyranoside for β-glucosidase; and 4-methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

Exemplary rare earth metals and metal isotopes suitable as a detectable label include, but are not limited to, lanthanide (III) isotopes such as 141Pr, 142Nd, 143Nd, 144Nd, 145Nd, 146Nd, 147Sm, 148Nd, 149Sm, 150Nd, 151Eu, 152Sm, 153Eu, 154Sm, 155Gd, 156Gd, 158Gd, 159Tb, 160Gd, 161Dy, 162Dy, 163Dy, 164Dy, 165Ho, 166Er, 167Er, 168Er, 169Tm, 170Er, 171Yb, 172Yb, 173Yb, 174Yb, 175Lu, and 176Yb. Metal isotopes can be detected, for example, using time-of-flight mass spectrometry (TOF-MS) (for example, Fluidigm Helios and Hyperion systems, fluidigm.com/systems; South San Francisco, CA).

Biotin-avidin (or biotin-streptavidin) is a well-known signal amplification system based on the fact that the two molecules have extraordinarily high affinity to each other, and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized horseradish peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. Pat. No. 8,658,361.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, OR); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NHS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

As disclosed herein, the methods provided herein can be used for concurrent detection of multiple target nucleic acids. In the case of using fluorophores as labels, the fluorophores to be used for detection of multiple target nucleic acids are selected so that each of the fluorophores are distinguishable and can be detected concurrently in the fluorescence microscope in the case of concurrent detection of target nucleic acids. Such fluorophores are selected to have spectral separation of the emissions so that distinct labeling of the target nucleic acids can be detected concurrently. Methods of selecting suitable distinguishable fluorophores for use in methods of the disclosure are known in the art (see, for example, Johnson and Spence, “Molecular Probes Handbook, a Guide to Fluorescent Probes and Labeling Technologies,”11th ed., Life Technologies (2010)).

Methods such as microscopy, cytometry (e.g., mass cytometry, cytometry by time of flight (CyTOF), flow cytometry), or spectroscopy can be utilized to visualize chromogenic, fluorescent, or metal detectable signals associated with the respective target nucleic acids. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of nucleic acid targets in the same sample.

As disclosed herein, the label can be designed such that the labels are optionally cleavable. As used herein, a cleavable label refers to a label that is attached or conjugated to a label probe so that the label can be removed, for example, in order to use the same label in a subsequent round of labeling and detecting of target nucleic acids. Generally, the labels are conjugated to the label probe by a chemical linker that is cleavable. Methods of conjugating a label to a label probe so that the label is cleavable are known to those skilled in the art (see, for example, Hermanson, Bioconjugate Techniques, Academic Press, San Diego (1996); Daniel et al., BioTechniques 24(3):484-489 (1998)). One particular system of labeling oligonucleotides is the FastTag™ system (Daniel et al., supra, 1998; Vector Laboratories, Burlinghame CA). Various cleavable moieties can be included in the linker so that the label can be cleaved from the label probe. Such cleavable moieties include groups that can be chemically, photochemically or enzymatically cleaved. Cleavable chemical linkers can include a cleavable chemical moiety, such as disulfides, which can be cleaved by reduction, glycols or diols, which can be cleaved by periodate, diazo bonds, which can be cleaved by dithionite, esters, which can be cleaved by hydroxylamine, sulfones, which can be cleaved by base, and the like (see Hermanson, supra, 1996). One particularly useful cleavable linker is a linker containing a disulfide bond, which can be cleaved by reducing the disulfide bond. In other embodiments, the linker can include a site for cleavage by an enzyme. For example, the linker can contain a proteolytic cleavage site. Generally, such a cleavage site is for a sequence-specific protease. Such proteases include, but are not limited to, human rhinovirus 3C protease (cleavage site LEVLFQ/GP), enterokinase (cleavage site DDDDK/), factor Xₐ (cleavage site IEGR/), tobacco etch virus protease (cleavage site ENLYFQ/G), and thrombin (cleavage site LVPR/GS) (see, for example, Oxford Genetics, Oxford, UK). Another cleavable moiety can be, for example, uracil-DNA (DNA containing uracil), which can be cleaved by uracil-DNA glycosylase (UNG) (see, for example, Sidorenko et al., FEBS Lett. 582(3):410-404 (2008)).

The cleavable labels can be removed by applying an agent, such as a chemical agent or light, to cleave the label and release it from the label probe. As discussed above, useful cleaving agents for chemical cleavage include, but are not limited to, reducing agents, periodate, dithionite, hydroxylamine, base, and the like (see Hermanson, supra, 1996). One useful method for cleaving a linker containing a disulfide bond is the use of tris(2-carboxyethyl)phosphine (TCEP) (see Moffitt et al., Proc. Natl. Acad. Sci. USA 113:11046-11051 (2016)). In one embodiment, TCEP is used as an agent to cleave a label from a label probe.

In some embodiments, the method for detecting a target nucleic acid in a cell provided herein comprises a pretreatment step before hybridization of the target probe(s). In some embodiments, the pretreatment step comprises a blocking step where certain blocking agent(s) is/are applied to block certain endogenous components of the cell thus reducing assay background. As described further herein, a blocking step can include contacting a biological sample with a composition comprising a plurality of RNA blocking molecules described herein. In accordance with these methods, the use of RNA blocking molecules of the present disclosure enhance signal efficiency so that the detection of the target nucleic acid using a target probe or set of target probes is improved compared to not using the RNA blocking molecules.

In some embodiments, various other blocking agents can be used in addition to the RNA blocking molecules of the present disclosure. For example, hydrogen peroxide is a blocking agent when horseradish peroxidase (HRP) is used as detection enzyme in the later steps. Hydrogen peroxide is added to inactivate the endogenous HRP activity in the sample, thus reducing assay background. In a specific embodiment, this blocking step is added as the first step in the pretreatment right after deparaffinization. In some embodiments, the pretreatment step comprises an epitope retrieval step, where certain epitope retrieval buffer(s) can be added to unmask the target nucleic acid. In some embodiments, the epitope retrieval step comprises heating the sample. In some embodiments, the epitope retrieval step comprises heating the sample to 50° C. to 100° C. In one embodiment, the epitope retrieval step comprises heating the sample to about 88° C. In some embodiments, the pretreatment step comprises a permeabilization step to retain the nucleic acid targets in the cell and to permit the target probe(s), signal-generating complex, etc. to enter the cell. In some embodiments, the permeabilization step comprises a digestion with a protease. Detergents (e.g., Triton X-100 or SDS) and Proteinase K can also be used to increase the permeability of the fixed cells. Detergent treatment, usually with Triton X-100 or SDS, is frequently used to permeate the membranes by extracting the lipids. Proteinase K is a nonspecific protease that is active over a wide pH range and is not easily inactivated. It is used to digest proteins that surround the target mRNA. Optimal concentrations and durations of treatment can be experimentally determined as is known in the art. A cell washing step can follow, to remove the dissolved materials produced in the any steps in the pretreatment step. In some embodiments, the sample is in a formalin-fixed paraffin embedded tissue, a deparaffinization step is needed, when paraffin is removed.

In some embodiments, the method for detecting a target nucleic acid in a cell provided herein comprises a post-fixation step at certain timing. In one embodiment, the post-fixation step is (i) after the first fixation step; and (ii) prior to applying at least one set of one or more target probe(s) capable of hybridizing to the target nucleic acid. In one embodiment, the post-fixation step is (i) after the first fixation step; and (ii) prior to pretreatment step described in the immediately preceding paragraph. In one embodiment, the post-fixation step is (i) after the first fixation step; and (ii) prior to the blocking step described in the immediately preceding paragraph. In one embodiment, the post-fixation step is (i) after the first fixation step; and (ii) prior to the epitope retrieval step described in the immediately preceding paragraph. In one embodiment, the post-fixation step is (i) after the first fixation step; and (ii) prior to the permeabilization step described in the immediately preceding paragraph. In one embodiment, the post-fixation step is (i) after the deparaffinization step; and (ii) prior to applying at least one set of one or more target probe(s) capable of hybridizing to the target nucleic acid. In one embodiment, the post-fixation step is (i) after the deparaffinization step; and (ii) prior to pretreatment step described in the immediately preceding paragraph. In one embodiment, the post-fixation step is (i) after the deparaffinization step; and (ii) prior to the blocking step described in the immediately preceding paragraph. In one embodiment, the post-fixation step is (i) after the deparaffinization step; and (ii) prior to the epitope retrieval step described in the immediately preceding paragraph. In one embodiment, the post-fixation step is (i) after the deparaffinization step; and (ii) prior to the permeabilization step described in the immediately preceding paragraph.

The methods provided herein have several applications in research and diagnostics (Hanna et al., Frontiers in Genetics, 10, 1-6, 2019; Watts et al., Journal of Pathology, 226(2), 365-379, 2012). The methods provided herein can improve our understanding of small nucleic acids, including sncRNAs, miRNAs, siRNAs, piRNAs, and ASOs, in their native context and their associated gene regulatory networks that are involved various of healthy stages and disease stages.

In some embodiments, the methods provided herein can detect small RNAs with spatial and temporal resolution. In one embodiment, the methods provided herein can be used for identification of tissues and cell types. In one embodiment, the methods provided herein can be used for identification of different stages of development. In one embodiment, the methods provided herein can be used for characterization of adult tissue.

In some embodiments, the methods provided herein can be used to detect altered small RNA expression or the presence of pathogen-associated small RNAs. In one embodiment, the methods provided herein can be used for diagnosing a disease or disorder. In one embodiment, the methods provided herein can be used for diagnosing pathogen.

In some embodiments, the methods provided herein are to monitor the effectiveness of a small RNA-based therapy. In one embodiment, the methods provided herein are to monitor the effectiveness of siRNA-based therapy. In one embodiment, the methods provided herein are to monitor the effectiveness of ASO-based therapy. In some embodiments, the methods provided herein are to determine the effectiveness of a small RNA-based therapy. In one embodiment, the methods provided herein are to determine the effectiveness of siRNA-based therapy. In one embodiment, the methods provided herein are to determine the effectiveness of ASO-based therapy.

In specific embodiments, the method can be used for detecting the presence of the siRNAs following the delivery of the siRNAs into disease models. In specific embodiments, the method can be used for localizing the siRNAs following the delivery of the siRNAs into disease models. In specific embodiments, the method can be used for quantifying the siRNAs following the delivery of the siRNAs into disease models. In specific embodiments, the method can be used for quantifying the RNAs that the siRNAs target following the delivery of the siRNAs into disease models.

In specific embodiments, the method can be used for detecting the presence of the ASOs following the delivery of the ASOs into disease models. In specific embodiments, the method can be used for localizing the ASOs following the delivery of the ASOs into disease models. In specific embodiments, the method can be used for quantifying the ASOs following the delivery of the ASOs into disease models. In specific embodiments, the method can be used for quantifying the RNAs that the ASOs target following the delivery of the ASOs into disease models.

### A Kit for In Situ Detection of a Target Nucleic Acid

Embodiments of the present disclosure also include a kit comprising any of the RNA blocking molecules described herein. The kit can also include any of the components described herein for performing an in situ hybridization reaction. In some embodiments, the kit comprises at least one target probe that specifically hybridizes to a probe-targeting region of a target RNA molecule. In some embodiments, kit further comprises one or more target probe sets, and each target probe set comprises a pair of target probes that specifically hybridize to a probe-targeting region of a target RNA molecule. In some embodiments, the kit comprises a reagent for permeabilizing cells, a cross-linking reagent, and/or a protease. In some embodiments, the kit comprises instructions for performing an in situ hybridization reaction.

In some embodiments, the kit comprises one or more components of a signal generating complex. In some embodiments, the components of a signal generating complex include: (i) at least one label and at least one label probe, wherein the at least one label probe is capable of hybridizing to the at least one target probe; or (ii) at least one label, at least one label probe, and at least one amplifier hybridized to the at least one label probe and capable of hybridizing to the at least one target probe; or (iii) at least one label, at least one label probe, at least one amplifier hybridized to the at least one label probe, and at least one preamplifier hybridized to the at least one amplifier and capable of hybridizing to the target probe.

Embodiments of the present disclosure also include a composition comprising any of the RNA blocking molecules described herein. In some embodiments, the composition comprises a hybridization buffer. In some embodiments, the composition comprises a reagent for permeabilizing cells, a cross-linking reagent, and/or a protease. In some embodiments, the composition comprises a biological sample. In some embodiments, the biological sample is a tissue specimen or is derived from a tissue specimen. In some embodiments, the biological sample is a blood sample or is derived from a blood sample. In some embodiments, the biological sample is a cytological sample or is derived from a cytological sample. In some embodiments, the biological sample is cultured cells or a sample containing exosomes. In some embodiments, the composition comprises at least one target probe that specifically hybridizes to a probe-targeting region of a target RNA molecule in the biological sample.

In some embodiments, the composition comprises one or more components of a signal generating complex. In some embodiments, the components of a signal generating complex include: (i) at least one label and at least one label probe, wherein the at least one label probe is capable of hybridizing to the at least one target probe; or (ii) at least one label, at least one label probe, and at least one amplifier hybridized to the at least one label probe and capable of hybridizing to the at least one target probe; or (iii) at least one label, at least one label probe, at least one amplifier hybridized to the at least one label probe, and at least one preamplifier hybridized to the at least one amplifier and capable of hybridizing to the target probe.

In some embodiments, the kit comprises an agent used for fixing a biological sample. In some embodiments, the kit includes a fixative(s) that is suitable for preserving nucleic acids. In one embodiment, the fixative is FineFix (see Kothmaier et al., Arch. Pathol. Lab. Med. 135:744-752, 2011). In one embodiment, the fixative is Glyo-fix (see Lykidis et al., Nucleic Acids Res. 35:e85, 2007). In one embodiment, the fixative is Histochoice (see Vince et al., Anal. Cell. Pathol. 15:119-129, 1997). In one embodiment, the fixative is HOPE (see Kothmaier et al., Arch. Pathol. Lab. Med. 135:744-752, 2011). In one embodiment, the fixative is Neo-Fix (see Paavilainen et al., Histochem. Cytochem.: Official J. Histochem. Soc. 58:237-246, 2010). In one embodiment, the fixative is the PAXgene Tissue System (see Nietner et al., Int. J. Pathol. 461:259-269, 2012). In one embodiment, the fixative is RCL2 (see van Essen et al., Clin. Pathol.63:1090-1094, 2010). In one embodiment, the fixative is Streck's Tissue Fixative (see Burns et al., Histochem. Cytochem. 57:257-264, 2009). In one embodiment, the fixative is UMFIX (see Nadji et al., Appl. Immunohistochem. Mol. Morphol. 13:277-282, 2005). In one embodiment, the fixative is Z7 (see Lykidis et al., Nucleic Acids Res. 35:e85, 2007). In one embodiment, the fixative is ZBF (see Paavilainen et al., Histochem. Cytochem.: Official J. Histochem. Soc. 58:237-246, 2010).

In some embodiments, the kit provided herein comprises an aldehyde-containing fixative. In one embodiment, the aldehyde-containing fixative in the kit is formaldehyde. In one embodiment, the aldehyde-containing fixative in the kit is glutaraldehyde. In one embodiment, the aldehyde-containing fixative in the kit is Bouin's fixative, which is a solution of picric acid, formaldehyde, and acetic acid. In one embodiment, the aldehyde-containing fixative in the kit is a mixture of formaldehyde and glutaraldehyde. In one embodiment, the aldehyde-containing fixative in the kit is FAA, which is a solution of ethanol, acetic acid, and formaldehyde. In one embodiment, the aldehyde-containing fixative in the kit is periodate-lysine-paraformaldehyde (PLP), which is a solution of paraformaldehyde, L-lysine, and INaO₄. In one embodiment, the aldehyde-containing fixative in the kit is phosphate buffered formalin (PBF). In one embodiment, the aldehyde-containing fixative in the kit is formal calcium, which is a solution of formaldehyde and calcium chloride. In one embodiment, the aldehyde-containing fixative in the kit is formal saline, which is a solution of formaldehyde and sodium chloride. In one embodiment, the aldehyde-containing fixative in the kit is zinc formalin, which is a solution of formaldehyde and zinc sulphate. In one embodiment, the aldehyde-containing fixative in the kit is Helly's fixative, which is a solution of formaldehyde, potassium dichromate, sodium sulphate, and mercuric chloride. In one embodiment, the aldehyde-containing fixative in the kit is Hollande's fixative, which is a solution of formaldehyde, copper acetate, picric acid, and acetic acid. In one embodiment, the aldehyde-containing fixative in the kit is Gendre's solution, which is a solution of formaldehyde, ethanol, picric acid, and acetic acid glacial. In one embodiment, the aldehyde-containing fixative in the kit is alcoholic formalin, which is a solution of formaldehyde, ethanol, and calcium acetate. In one embodiment, the aldehyde-containing fixative in the kit is formol acetic alcohol, which is a solution of formaldehyde, acetic acid glacial, and ethanol. In one embodiment, the aldehyde-containing fixative in the kit is a mixture of fixatives, wherein at least one fixative of the mixture is formaldehyde or glutaraldehyde. In one embodiment, the aldehyde-containing fixative in the kit is fixatives that are not used at the same time but consecutively, wherein at least one fixative is formaldehyde or glutaraldehyde.

In some embodiments, the aldehyde-containing fixative in the kit provided herein comprises about 5% to about 50% formaldehyde. In other embodiments, the aldehyde-containing fixative comprises about 10% to about 40% formaldehyde. In yet other embodiments, the aldehyde-containing fixative comprises about 12% to about 37% formaldehyde.

In some embodiments, the aldehyde-containing fixative in the kit provided herein comprises various concentrations of formaldehyde. In one embodiment, the aldehyde-containing fixative comprises about 5% formaldehyde. In one embodiment, the aldehyde-containing fixative comprises about 6% formaldehyde. In one embodiment, the aldehyde-containing fixative comprises about 7% formaldehyde. In one embodiment, the aldehyde-containing fixative comprises about 8% formaldehyde. In one embodiment, the aldehyde-containing fixative comprises about 9% formaldehyde. In one embodiment, the aldehyde-containing fixative comprises about 10% formaldehyde. In one embodiment, the aldehyde-containing fixative comprises about 11% formaldehyde. In one embodiment, the aldehyde-containing fixative comprises about 12% formaldehyde. In one embodiment, the aldehyde-containing fixative comprises about 13% formaldehyde. In one embodiment, the aldehyde-containing fixative comprises about 14% formaldehyde. In one embodiment, the aldehyde-containing fixative comprises about 15% formaldehyde. In one embodiment, the aldehyde-containing fixative comprises about 16% formaldehyde. In one embodiment, the aldehyde-containing fixative comprises about 17% formaldehyde. In one embodiment, the aldehyde-containing fixative comprises about 18% formaldehyde. In one embodiment, the aldehyde-containing fixative comprises about 19% formaldehyde. In one embodiment, the aldehyde-containing fixative comprises about 20% formaldehyde. In one embodiment, the aldehyde-containing fixative comprises about 30% formaldehyde. In one embodiment, the aldehyde-containing fixative comprises about 35% formaldehyde. In one embodiment, the aldehyde-containing fixative comprises about 40% formaldehyde. In one embodiment, the aldehyde-containing fixative comprises about 50% formaldehyde.

In some embodiments, the kit further comprises a tool for obtaining a biological sample from a subject. In certain embodiments, the biological sample is a tissue specimen or is derived from a tissue specimen. In certain embodiments, the biological sample is a blood sample or is derived from a blood sample. In certain embodiments, the biological sample is a cytological sample or is derived from a cytological sample.

In some embodiments, the target nucleic acid is DNA. In some embodiments, the target nucleic acid is RNA. In some embodiments, the target nucleic acid is long RNA. In some embodiments, the target nucleic acid is short RNA. In some embodiments, the target nucleic acid is RNA comprising less than 100 nucleotides. In other embodiments, the target nucleic acid is RNA comprising less than 50 nucleotides. In other embodiments, the target nucleic acid is RNA comprising between 15 and 40 nucleotides. In some embodiments, the target nucleic acid is sncRNA. In other embodiments, the target nucleic acid is miRNA, siRNA, piRNA, or ASO. In yet other embodiments, the target nucleic acid is an endogenous RNA or an exogenous RNA.

In a specific embodiment, the kit provided herein comprises agents for performing RNAscope® as described in more detail in, e.g., U.S. Pat. Nos. 7,709,198, 8,604,182, and 8,951,726. In some embodiments, the kit comprises at least one set of one or more target probe(s) capable of hybridizing to a target nucleic acid; a signal-generating complex capable of hybridizing to said set of one or more target probe(s), wherein said signal-generating complex comprises a label probe and a nucleic acid component capable of hybridizing to said set of one or more target probe(s). In some embodiments, the target probe(s) comprises a target (T) section and a label (L) section, wherein the T section is a nucleic acid sequence complementary to a section on the target nucleic acid and the L section is a nucleic acid sequence complementary to a section on the nucleic acid component of the signal-generating complex, and wherein the T sections of the one or more target probe(s) are complementary to non-overlapping regions of the target nucleic acid, and the L sections of the one or more target probe(s) are complementary to non-overlapping regions of the nucleic acid component of the generating complex.

In some embodiments, the kit further comprises signal-generating complex as described above, which may incudes label probe, amplifier, pre-amplifier, and/or pre-pre-amplifier. In some embodiments, the kit further comprises other agents or materials for performing RNA ISH, including fixing agents and agents for treating samples for preparing hybridization, agents for washing samples, and so on.

The kit may further comprise “packaging material” which refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits provided herein can include labels or inserts. Labels or inserts include information on a condition, disorder, disease, or symptom for which the kit component may be used for. Labels or inserts can include instructions for a clinician or for a subject to use one or more of the kit components in a method, treatment protocol, or therapeutic regimen. In some embodiments, the kit can be used for identification of tissues and cell types. In some embodiments, the kit can be used for identification of different stages of development. In some embodiments, the kit can be used for detection of clinical biomarkers for cancers. In some embodiments, the kit can be used for diagnosing a disease or disorder based on the expression of one or more altered small RNAs or the presence of pathogen-associated small RNAs. In some embodiments, the kit can be used for characterization of adult tissue. In some embodiments, the kit can be used for detection of clinical biomarkers for pathogen diagnosis. In some embodiments, the kit can be used for detection and characterization of small RNA-based therapies. In some embodiments, the kit can be used for confirmation of the initial efficiency of small RNA-based therapies. In some embodiments, the kit can be used to continue monitoring the efficiency of small RNA-based therapies. In some embodiments, the kit can be used for determining the efficiency of small RNA-based therapies. In some embodiments, the kit can be used for detecting the presence, localizing, and quantifying siRNAs. In some embodiments, the kit can be used for detecting the presence, localizing, and quantifying ASO molecules. In some embodiments, the kit can be used for detection and identification of pathogen-derived small RNAs.

### Image Processing

Embodiments of the present disclosure also include a method for enhancing detection of a target (e.g., a target nucleic acid). In some embodiments, the method includes an image processing method, such as the methods described in International Patent Application PCT/US22/24975, which is herein incorporated by reference. The method is implemented at least in part with a computer having corresponding instructions stored on a memory (i.e., a non-transitory computer readable medium). The final images, and in some embodiments the intermediate images, from the method are stored in a memory. In some embodiments, the memory is accessible by a network. In some embodiments, user input or instructions are receivable or accessible over the network.

The method includes imaging a sample with a target signal to create a probe image and imaging a sample with no target signal to create a background image (i.e., “blank image”). In some embodiments, a “blank image” is an image that includes RNA blocking molecules of the present disclosure. In some embodiments, a “blank image” is an image that does not include RNA blocking molecules of the present disclosure. In some embodiments, the imaging utilizes a fluorescent microscope coupled to a computer via a network. In some embodiments, the target signal is obtained by subjecting the sample to a fluorescent in situ hybridization assay and/or an immunofluorescence assay. In some embodiments, the background image with no target signal is obtained by removing the target signal from the sample (i.e., by a cleaving process). In other embodiments, the background image with no target signal is obtained before the assay is performed. In some embodiments, the target signal comprises a fluorescent label bound to a target nucleic acid. In other embodiments, the target signal comprises a fluorescent label bound to a target peptide or polypeptide.

The method can also include registering the probe image and the background image (e.g., with or without RNA blocking molecules). Potential background fluorescence discrepancy between the probe image and the background image creates spatial pattern mismatches that occur due to whole sample movement between different rounds of image acquisition. To remove such discrepancies, image registration techniques (e.g., phase correlation) are utilized. Robust image registration utilizes detection and matching of image features to compensate for any global sample movement (i.e., translation and rotation).

The method further includes modifying the background image (e.g., with or without RNA blocking molecules) to create an adjusted background image (e.g., transformed, intensity-adjusted blank image) based on at least one image metric. As explained further herein, the at least one image metric is a ratio factor, a multiplication factor, a local maximum value transform, and any other suitable metric. In some embodiments, the method includes a single image metric. In other embodiments, the method includes a combination of image metrics.

In some embodiments, the method further includes subtracting the adjusted background image (e.g., with or without RNA blocking molecules) from the probe image to create a final image comprising an enhanced target signal. In other words, the modified (i.e., transformed, adjusted, scaled, etc.) blank image is used in the subtracting step instead of the original blank image. In some embodiments, the enhanced target signal includes enhanced contrast. In some embodiments, the method includes displaying the final image on a display (e.g., a computer display). The final image may be saved to a memory and may be accessible by a user, for example, over a network. As such, the method provides improved signal detection in the presence of a background with tissue autofluorescence.

In some embodiments, the image metric is a ratio factor to account for intensity differences in background between the blank image and the probe image. Intensity differences can occur when image acquisition settings are different or from photobleaching during fluorophore excitation. To compensate for background intensity differences, the method includes determining a ratio factor that compares the overall background intensity of the probe image versus the blank image. First, the pixel locations of the probe are estimated. The probe locations in the probe image are estimated using, for example, the White Top Hat algorithm (Gonzalez & Woods, 2008, Digital Image Processing), bandpass filtering (Shenoi, 2006, Introduction to Digital Signal Processing and Filter Design), or any combination of suitable methods. After determining an estimated location of the target signals in the probe image, the pixels at the estimated probe locations are excluded from both the probe image and the blank image, resulting in background-pixel-only images (i.e., background-only images). In other words, the method includes removing the estimated location from the probe image to create a first background-only image and removing the estimated location from the blank image (background image) to create a second background-only image.

Following removal of the estimated probe locations from both images, the method includes determining a ratio factor. In other words, a statistical metric for both the probe-excluded blank image and the probe-excluded probe image is evaluated and incorporated into a ratio factor. The ratio factor is utilized in some embodiments to modify the background image to create an adjusted background image. In other words, modifying the background image to create an adjusted background image can include, in some embodiments, scaling the background image by the ratio factor.

In some embodiments, the at least one image metric is a ratio factor of the first background-only image and the second background-only image. For example, the ratio factor in some embodiments is a first intensity to a second intensity, with the first intensity determined from the first background-only image and the second intensity is determined from the second background-only image. In some embodiments, the first and second intensities used in the ratio factor are statistical metrics such as a statistical mean, median, or a combination of both for any portion of (including all) the intensity values in an image.

In some embodiments, the first intensity is the mean of a plurality of pixel intensity values in the first background-only image and the second intensity is the mean of a plurality of pixel intensity values in the second background-only image. In some embodiments, the mean is of all the pixel intensity values in the image. In other embodiments, the first intensity is the median of a plurality of pixel intensity values in the first background-only image, and the second intensity is the median of a plurality of pixel intensity values in the second background-only image. In some embodiments, the median is of all the pixel intensity values in the image. In another embodiment, the first intensity is the mean of a central approximately 80% of all the pixel intensity values (i.e., excluding the approximate top 10% and the approximate bottom 10%) in the first background-only image, and the second intensity is the mean of a central approximately 80% of all the pixel intensity values in the second background-only image.

In some embodiments, the image metric is a multiplication factor to account for potential local intensity differences between the blank image and the probe image. In particular, the method includes determining the multiplication factor. In some embodiments, the multiplication factor is within a range of approximately 1.0 to approximately 1.2. In other embodiments, the multiplication factor is within a range of approximately 1.0 to approximately 1.1. The multiplication factor is utilized in some embodiments to modify the background image to create an adjusted background image. In other words, modifying the background image to create an adjusted background image can include, in some embodiments, scaling the background image by the multiplication factor.

In some embodiments, the image metric is a local maximum value transform. In particular, the method includes transforming the blank image with a local maximum value transform. Even after global image registration, there may remain local background pattern mismatches that from, for example, image acquisition at different focal planes, or samples not firmly attached to the supporting material (e.g., glass slides) and partially moving between imaging sessions. To resolve this issue, local mismatches are compensated accordingly. In the illustrated embodiment, for each pixel in the blank image (“pixel of interest”), a neighborhood of a pre-defined radius surrounding the pixel of interest is searched. The search process will find the pixel of maximum intensity, and this maximum intensity is assigned to that pixel of interest. This searching procedure is performed for each pixel of interest, searching its neighborhood in the original blank image, to form a transformed blank image. As explained in greater detail herein, the transformed blank image can be used instead of the original blank image in the later subtracting step. In some embodiments, the pre-defined radius (“match distance”) is adjustable.

In some embodiments, the pre-defined radius used in the local maximum valve transform is within a range of approximately 0 to approximately 5 pixels. In other words, the local maximum value transform includes a search radius within a range of approximately 0 to 5 pixels. A pre-defined radius of 0 pixels is utilized, for example, when there is no noticeable local background pattern mismatch. In some embodiments, the search area is simplified to reduce computational time by using eight angularly equally spaced lines (i.e., 45 degrees apart), each with a single-pixel width, radiating from the pixel of interest.

In some embodiments, the image metric is a block-matching transform. In particular, the method, in some embodiments, includes a step to transform the blank image with a block-matching transform. In some embodiments, the block-matching transform is used in place of the local maximum value transform to resolve the issue of local mismatches. In some embodiments, a block (“block of interest”) is used with a pre-defined block size (e.g., a 3-pixel-by-3-pixel block). Each block in the blank image is compared with blocks of the same size in the probe image in nearby locations (i.e., within a pre-defined block search size). The search determines the nearby block that is most similar to the block of interest. A similarity metric is utilized to measure the similarity of the blocks, and the searched nearby block with the highest similarity metric is determined to be the target block. Then, the block of interest is moved to the corresponding location of the target block. In some embodiments, the similarity metric is a mean absolute difference, a sum of absolute difference, a mean squared difference, or a sum of squared difference, wherein the differences are the pixel intensity differences between the two blocks being compared. As such, the block-matching transform is performed for each block of interest, searching its corresponding neighborhood in the probe image and moving its location accordingly, to form a transformed blank image. In some embodiments, this transformed blank image is used instead of the original blank image in later subtracting steps.

In some embodiments, the pre-defined block size and the pre-defined block search size are adjustable. In some embodiments, the pre-defined block size used in the block-matching transform is within a range of approximately 1 to approximately 10 pixels. In other words, the block-matching transform includes a block size within a range of approximately 1 to 10 pixels. In some embodiments, the pre-defined block search size used in the block matching transform is within a range of approximately 1 to approximately 10 pixels. In other words, the block-matching transform includes a block search size within a range of approximately 1 to 10 pixels.

In some embodiments, the method for enhancing detection of a target includes any combination of the steps described herein, in various orders. In some embodiments, steps may be omitted. Further, the order of the steps may be reversed, altered, or performed simultaneously.

In at least one embodiment, the electronic-based aspects of the method may be implemented in software (e.g., stored on non-transitory computer-readable medium) executable by a computer with one or more processing units, such as a microprocessor and/or application specific integrated circuits (“ASICs”). Some embodiments may include hardware, software, and electronic components or modules. As such, it should be noted that a plurality of hardware and software-based devices, as well as a plurality of different structural components, may be utilized to implement the embodiments.

### EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### RNA Blocking Molecule Design and Evaluation

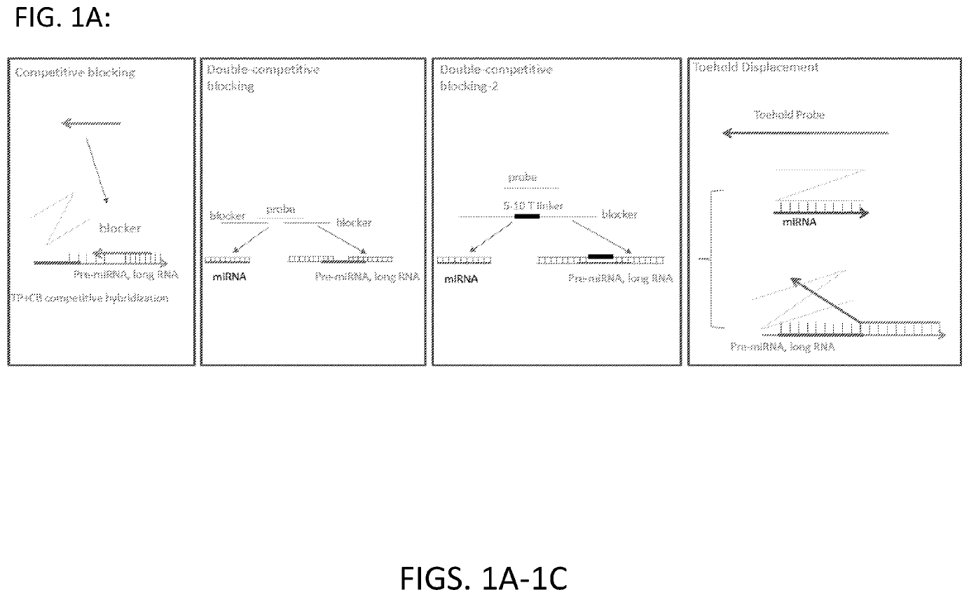

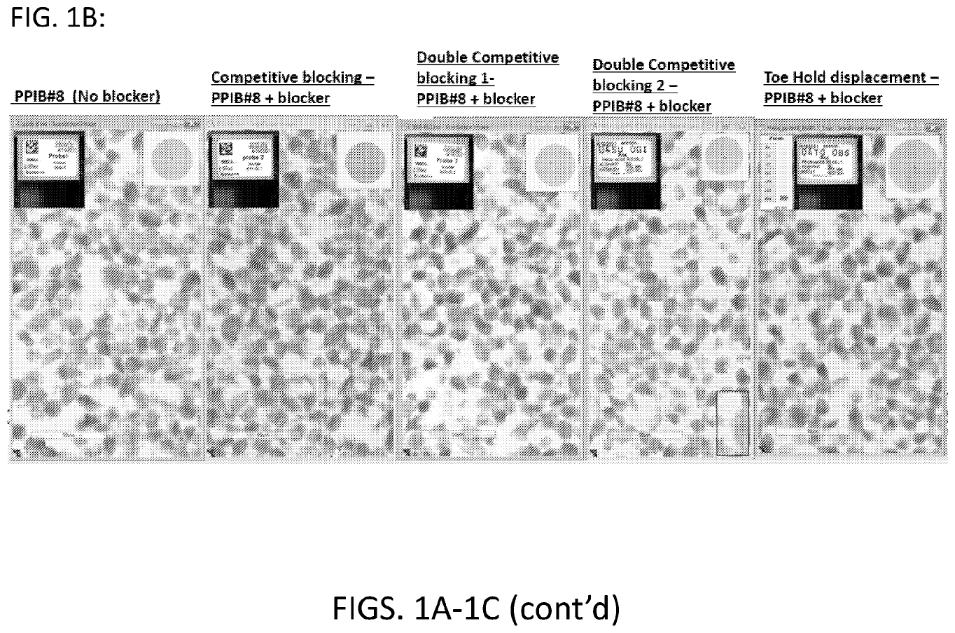

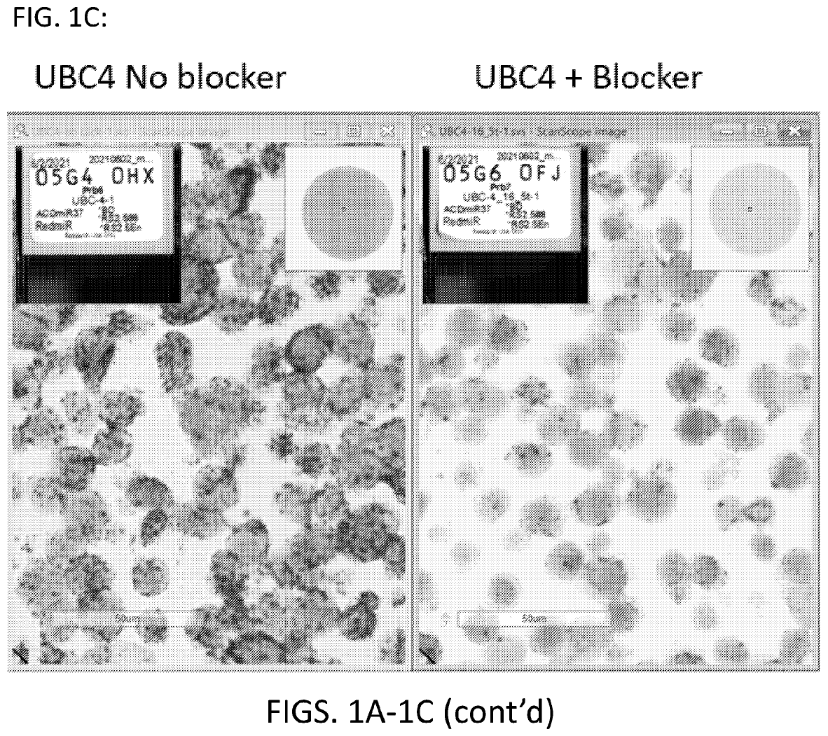

Various strategies for reducing off-target binding of target probes to non-specific nucleic acids in a sample, and concomitantly, for enhancing binding of target probes to their corresponding target nucleic acid in a sample, were designed and tested. FIG. 1A provides four design strategies that were evaluated, including (i) competitive blocking; (ii) double-competitive blocking; (iii) double-competitive blocking 2; and (iv) toehold displacement. To test the efficiency of each of the RNA blocking design strategies for blocking or reducing off-target binding of the probes, oligos were designed against a low expressing gene, PPIB (Peptidylprolyl Isomerase B; Accession No. NM_000942.4), which was chosen as a proxy off-target binding. Four different blocking strategies/designs were tested in order to determine reduction in signal from PPIB (FIG. 1B). As shown, although each of the four design strategies tested resulted in some reduction of off-target binding of the target probes; however, oligos with sequences complementary to the two sides of the probe design region that were connected with a T-linker (“Double Competitive Blocking 2” strategy) were most efficient in blocking signal from PPIB (FIG. 1B).

Similar oligos were designed and tested against a high-expressing gene, UBC. RNA blocking molecules designed using the same strategy (“Double Competitive Blocking 2” strategy) efficiently reduced the signal from UBC probe (FIG. 1C). RNA blocking molecule used in these experiments were 19 bp long and included 5′-O-methyl modifications.

### Effect of Blockers on Immature Pre- and Pri-miRNA21 and No Effect of Blocker on Target Signal

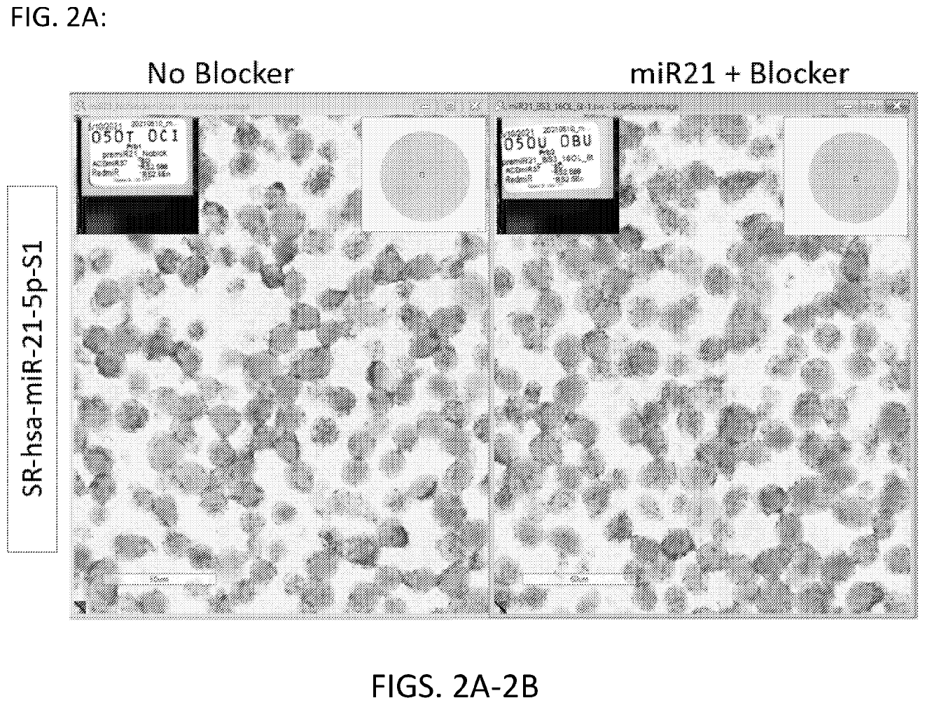

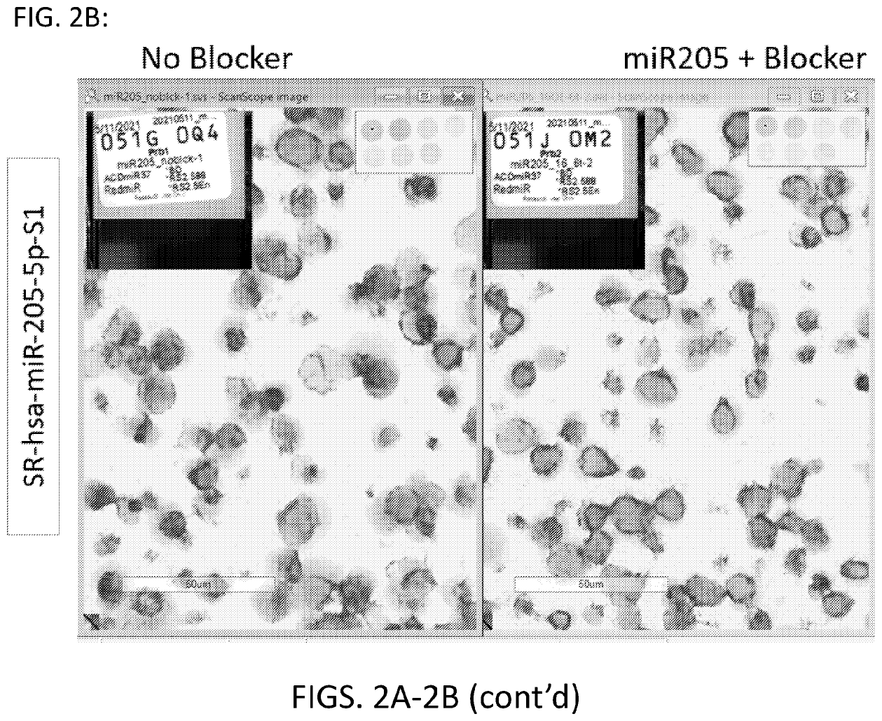

After determining effective designs for blockers, the effect of blockers on the ability to detect target miRNA was tested. Experiments were conducted to evaluate T-linker blockers against miRNA-21 and miRNA-205. As shown in FIGS. 2A and 2B, results demonstrated that RNA blocking molecules directed against pri-miR-21 and pre-miR-205 did not affect detection of miRNA-21 (FIG. 2A) and miRNA-205 (FIG. 2B), respectively.

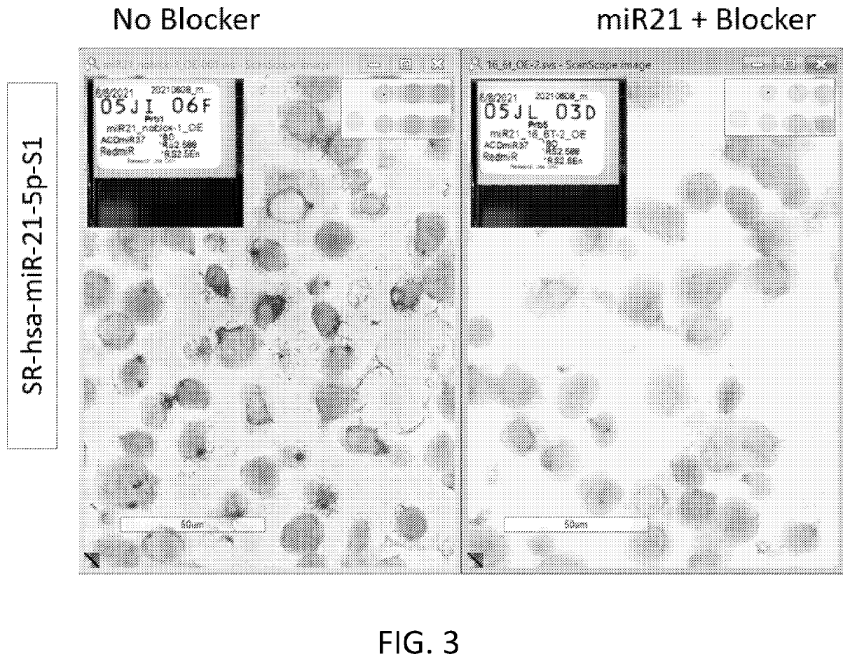

Additionally, experiments were conducted to evaluate RNA blocking molecules with RNAscope® probes designed to bind to both mature and long immature (pre- and pri-miRNAs). To test the efficiency of RNA blockers in blocking signal from longer targets, a model system was developed by transfecting HeLa cells with pre- and pri-miRNA. Pre- and pri-miR21 were transfected at 1 nM concentrations and reduction in signal was determined after addition of blockers. Presence of blockers reduced the signal in the over-expression cell pellet (FIG. 3). Experiments were also conducted to determine the effect of blockers on the mature miR-21 signal. HeLa cell pellet with endogenous levels of miR21 shows that blockers had no effect on probe binding efficiency, indicated by no-reduction in signal.

### Optimization of RNA Blocking Molecules

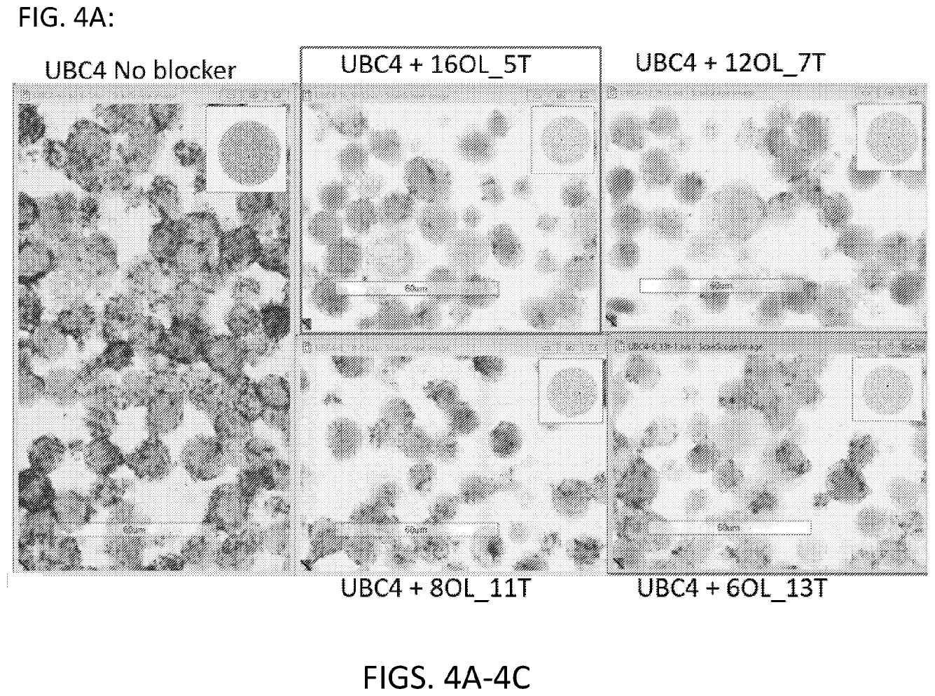

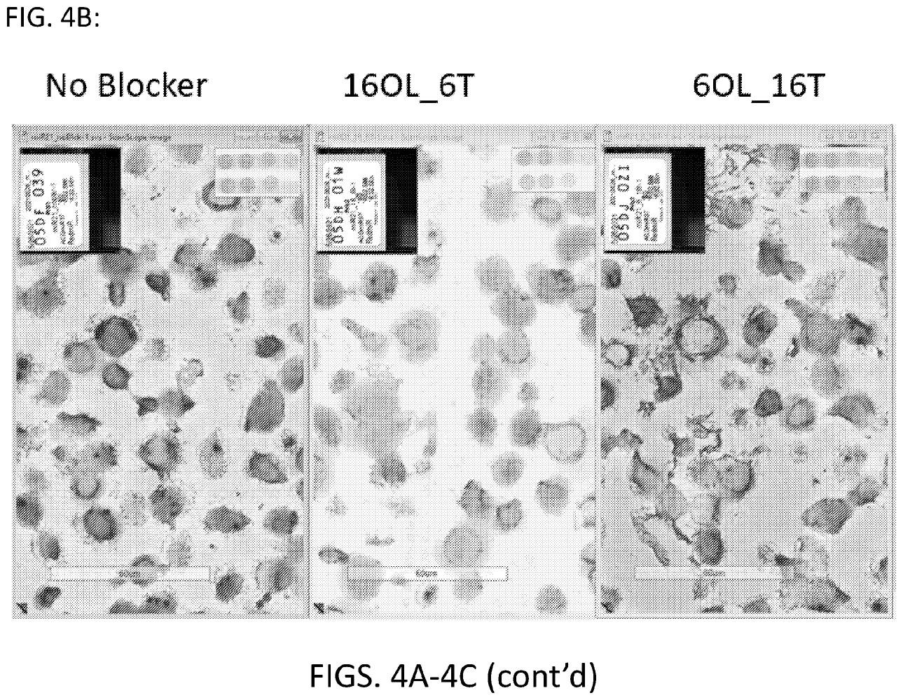

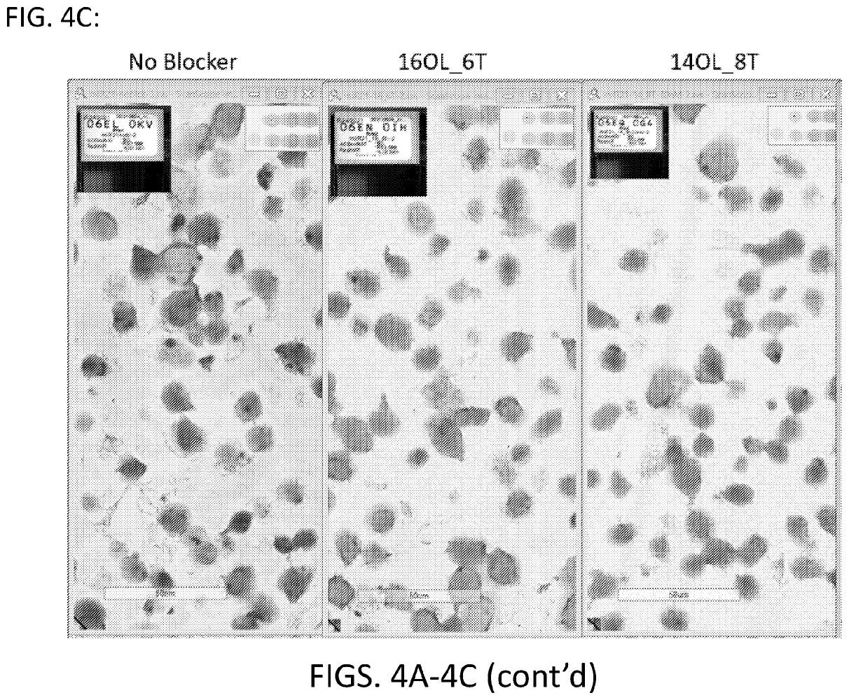

The probes used in miRNAscope® compositions and methods vary in length from about 16 to about 22 nucleotides. In accordance with these compositions and methods, various features of the RNA blockers of the present disclosure can be optimized, including but not limited to, the overlap length (probe-targeting region of the blockers) of the longer pre- and pri-miRNAs, the linker length that connects the two blockers, and the minimum concentration required for best efficiency. To develop a universal blocker design, varying overlap lengths were tested against UBC (Ubiquitin C; Accession No. NM_021009) and miR21 targets. Results indicated that longer overlap lengths of about 14 to about 16 nucleotides had higher blocking efficiency, as indicated by lower signal in blocker-added sample compared to no-blocker sample (FIG. 4A-4C).

Experiments were also conducted using varying lengths of the linker, while keeping the overlap length constant. Blockers with varying linker lengths (e.g., T linker lengths) were designed against UBC and miR21, while the overlap length was 14 or 16 nucleotides total. Blockers with shorter T-linker length showed higher efficiency in blocking non-specific signal as compared to the longer T-linker blockers (FIG. 5A), with linkers of about 1-3 nucleotides in length being most efficient (FIGS. 5B and 5C). Results also demonstrated that T linkers are somewhat more efficient that linkers composed of other nucleotides (FIG. 5D), though this may depend on the nucleotides that make up the probe targeting region of the target RNA and their complementarity to the nucleotides in the linker region.

Further, varying blocker concentrations were tested to determine the optimal concentration of the blockers with higher efficiency. The blockers for UBC and miR21 were tested at 10, 20, 30, 40 and 50 nM concentration (FIG. 5E). Blockers at 20 and 30 nM showed highest blocking efficiency. These data suggest that one representative optimal RNA blocker design includes an overlap region of about 14-16 nucleotides (i.e., probe targeting region), with a linker length that is about 2-3 nucleotides, and a concentration from about 20 nM to about 30 nM (FIG. 5F).

What is claimed is:

1. An RNA blocking molecule comprising at least one RNA blocking domain comprising a non-probe-targeting region and a probe-targeting region, wherein the regions are contiguous.

2. The RNA blocking molecule of claim 1, wherein the non-probe-targeting region of the RNA blocking domain is complementary to a non-probe-targeting region of a target RNA molecule.

3. The RNA blocking molecule of claim 1, wherein the probe-targeting region of the RNA blocking domain is complementary to a portion of a probe-targeting region of a target RNA molecule.

4. The RNA blocking molecule of claim 2 or claim 3, wherein the target RNA molecule is an mRNA molecule, a microRNA (miRNA) molecule, a small non-coding RNA (sncRNA) molecule, a PIWI-interacting RNA (piRNA) molecule, a small interfering RNA (siRNA) molecule, and/or antisense oligonucleotides (ASO).

5. The RNA blocking molecule of any one of claims 1 to 4, wherein the non-probe-targeting region of a target mRNA comprises a non-coding region, and wherein the probe-targeting region of the target mRNA comprises a coding region.

6. The RNA blocking molecule of any one of claims 1 to 4, wherein the non-probe-targeting region of a target miRNA comprises a region that is not present in the corresponding mature miRNA molecule, and wherein the probe-targeting region of the target miRNA comprises a region that is present in the corresponding mature miRNA molecule.

7. The RNA blocking molecule of any one of claims 1 to 6, wherein the non-probe-targeting region of the RNA blocking domain is from 2 to 50 nucleotides in length.

8. The RNA blocking molecule of any one of claims 1 to 7, wherein the probe-targeting region of the RNA blocking domain is from 2 to 20 nucleotides in length.

9. The RNA blocking molecule of any one of claims 1 to 8, further comprising a second RNA blocking domain comprising a second non-probe-targeting region and a second probe-targeting region, wherein the second regions are contiguous.

10. The RNA blocking molecule of any one of claims 1 to 9, wherein the first and the second RNA blocking domains are joined by a linker region.

11. The RNA blocking molecule of any one of claims 1 to 10, wherein the first and the second RNA blocking domains comprise the same number of nucleotides.

12. The RNA blocking molecule of any one of claims 1 to 11, wherein the non-probe-targeting regions of the first and the second RNA blocking domains comprise the same number of nucleotides.

13. The RNA blocking molecule of any one of claims 1 to 12, wherein the probe-targeting regions of the first and the second RNA blocking domains comprise the same number of nucleotides.

14. The RNA blocking molecule of any one of claims 10 to 13, wherein the linker region is from 1 to 10 nucleotides in length.

15. The RNA blocking molecule of any one of claims 10 to 14, wherein the linker region is from 2 to 8 nucleotides in length.

16. The RNA blocking molecule of any one of claims 10 to 15, wherein the linker region is from 2 to 5 nucleotides in length.

17. The RNA blocking molecule of any one of claims 10 to 16, wherein the linker region comprises at least two types of nucleotides.

18. The RNA blocking molecule of any one of claims 10 to 17, wherein the linker region comprises one type of nucleotide.

19. The RNA blocking molecule of claim 18, wherein the linker region consists of thymine nucleotides.

20. The RNA blocking molecule of claim 21, wherein the linker region consists of 1 to 10 thymine nucleotide(s).

21. The RNA blocking molecule of any one of claims 10 to 20, wherein the linker region comprises at least one nucleotide that is non-complementary to a portion of a probe-targeting region of a target RNA molecule.

22. The RNA blocking molecule of any one of claims 10 to 21, wherein the linker region comprises from 1 to 10 nucleotides that are non-complementary to a portion of a probe-targeting region of a target RNA molecule.

23. The RNA blocking molecule of any one of claims 10 to 21, wherein from about 20% to about 80% of the nucleotides comprising the linker region are non-complementary with a portion of a probe-targeting region of a target RNA molecule.

24. The RNA blocking molecule of any one of claims 10 to 23, wherein the total length of the RNA blocking molecule is from 15 to 100 nucleotides.

25. A kit comprising any of the RNA blocking molecules of claims 1 to 24.

26. The kit of claim 25, wherein the kit further comprises at least one target probe that specifically hybridizes to a probe-targeting region of a target RNA molecule.

27. The kit of claim 25, wherein the kit further comprises one or more target probe sets, wherein each target probe set comprises a pair of target probes that specifically hybridize to a probe-targeting region of a target RNA molecule.

28. The kit of any one of claims 25 to 27, wherein the kit comprises a reagent for permeabilizing cells, a cross-linking reagent, and/or a protease.

29. The kit of any one of claims 25 to 28, wherein the kit comprises one or more components of a signal generating complex.

30. The kit of claim 29, wherein the one or more components of a signal generating complex comprises:
(i) at least one label and at least one label probe, wherein the at least one label probe is capable of hybridizing to the at least one target probe; or
(ii) at least one label, at least one label probe, and at least one amplifier hybridized to the at least one label probe and capable of hybridizing to the at least one target probe; or
(iii) at least one label, at least one label probe, at least one amplifier hybridized to the at least one label probe, and at least one preamplifier hybridized to the at least one amplifier and capable of hybridizing to the target probe.

31. The kit of any one of claims 25 to 30, wherein the kit comprises instructions for performing an in situ hybridization reaction.

32. A composition comprising any of the RNA blocking molecules of claims 1 to 24.

33. The composition of claim 32, wherein the composition comprises a hybridization buffer.

34. The composition of any one of claims 32 to 33, wherein the composition comprises a reagent for permeabilizing cells, a cross-linking reagent, and/or a protease.

35. The composition of any one of claims 32 to 34, wherein the composition comprises a biological sample.

36. The composition of claim 35, wherein the biological sample is a tissue specimen or is derived from a tissue specimen.

37. The composition of claim 35, wherein the biological sample is a blood sample or is derived from a blood sample.

38. The composition of claim 35, wherein the biological sample is a cytological sample or is derived from a cytological sample.

39. The composition of claim 35, wherein the biological sample is cultured cells or a sample containing exosomes.

40. The composition of any one of claims 33 to 39, wherein the composition comprises at least one target probe that specifically hybridizes to a probe-targeting region of a target RNA molecule in the biological sample.

41. The composition of any one of claims 33 to 40, wherein the composition comprises one or more components of a signal generating complex.

42. The composition of claim 41, wherein the one or more components of a signal generating complex comprises:
(i) at least one label and at least one label probe, wherein the at least one label probe is capable of hybridizing to the at least one target probe; or
(ii) at least one label, at least one label probe, and at least one amplifier hybridized to the at least one label probe and capable of hybridizing to the at least one target probe; or
(iii) at least one label, at least one label probe, at least one amplifier hybridized to the at least one label probe, and at least one preamplifier hybridized to the at least one amplifier and capable of hybridizing to the target probe.

43. A method for performing an in situ hybridization reaction using any of the RNA blocking molecules of any one of claims 1 to 24, any of the kits of claims 25 to 31, or any of the compositions of any one of claims 32-42.

44. A method of enhancing signal efficiency in an in situ hybridization reaction, the method comprising:
contacting a biological sample comprising a target RNA molecule with any of the RNA blocking molecules of any one of claims 1 to 24;
contacting the biological sample with at least one target probe that specifically hybridizes to a probe-targeting region of the target RNA molecule; and
contacting the biological sample with a signal generating complex to detect the target RNA molecule.

45. The method of claim 44, wherein signal efficiency for the target RNA molecule is enhanced as compared to an in situ hybridization reaction that does not comprise contacting the biological sample with the RNA blocking molecules.

46. The method of claim 44 or claim 45, wherein enhancing signal efficiency comprises reducing binding of the target probe to non-target RNA molecules in the sample.

47. The method of any one of claims 44 to 46, wherein the target RNA molecule is an mRNA molecule, a microRNA (miRNA) molecule, a small non-coding RNA (sncRNA) molecule, a PIWI-interacting RNA (piRNA) molecule, a small interfering RNA (siRNA) molecule, and/or antisense oligonucleotides (ASO).

48. The method of any one of claims to 47, wherein the biological sample is a tissue specimen or is derived from a tissue specimen, a blood sample or is derived from a blood sample, or a cytological sample or is derived from a cytological sample.

49. The method of any one of claims 44 to 48, wherein the signal generating complex comprises:
(i) at least one label and at least one label probe, wherein the at least one label probe is capable of hybridizing to the at least one target probe; or
(ii) at least one label, at least one label probe, and at least one amplifier hybridized to the at least one label probe and capable of hybridizing to the at least one target probe; or
(iii) at least one label, at least one label probe, at least one amplifier hybridized to the at least one label probe, and at least one preamplifier hybridized to the at least one amplifier and capable of hybridizing to the target probe.

50. The method of any one of claims 44 to 49, wherein the method comprises treating the biological sample with a reagent for permeabilizing cells, a cross-linking reagent, and/or a protease.
